(19) United States
(12) Patent Application Publication
(10) Pub. No.: US 2026/0325670 A1
(43) Pub. Date: Oct. 22, 2026

(54) TARGETED ENCAPSULATION STRATEGIES FOR TREATMENT MODALITIES

(71) Applicant: NUTARIA LTD., Beer Sheba (IL)

(72) Inventor: Omer PORAT, Beer Sheva (IL)

(21) Appl. No.: 19/722,888

(22) Filed: Jun. 28, 2026

Related U.S. Application Data
(63) Continuation-in-part of application No. 18/601,302, filed on Mar. 11, 2024.
Application No. 18/601,302 is a continuation of application No. PCT/IL2022/051014, filed on Sep. 22, 2022.
(60) Provisional application No. 63/248,448, filed on Sep. 25, 2021.

(51) Int. Cl. A61K 47/69; A61K 31/05; A61K 31/121; A61K 31/122; A61K 31/352; A61K 31/355; A61K 47/54; A61K 47/55; A61P 25/16

(52) U.S. Cl. CPC A61K 47/6911; A61K 31/05; A61K 31/121; A61K 31/122; A61K 31/352; A61K 31/355; A61K 47/545; A61K 47/55; A61P 25/16

(57) ABSTRACT

The invention provides a composition for delivering an active pharmaceutical ingredient (API) and/or active nutraceutical ingredient (ANI) to the basal ganglia of a subject in need thereof comprising a plurality of nanoparticles surface decorated with caffeine, wherein the nanoparticles comprising: at least one API or ANI, at least one emulsifier, at least one emulsifier stabilizer, at least one water miscible solvent, and at least one solid lipid matrix core; and a pharmaceutically or nutraceutically acceptable carrier. The invention further provides methods for delivering active ingredients to the to the basal ganglia of a subject in need thereof and methods for treating Parkinson's disease.

### CROSS REFERENCE TO RELATED APPLICATIONS

This application is a Continuation In Part of U.S. patent application Ser. No. 18/601,302, which is a Bypass Continuation of PCT Patent Application No. PCT/IL2022/051014 having International filing date of Sep. 22, 2022, which claims the benefit of priority of U.S. Provisional Patent Application No. 63/248,448, filed Sep. 25, 2021, the contents of which are all incorporated herein by reference in their entirety.

### FIELD OF THE INVENTION

This invention is in the field of targeted and enriched nutraceutical-based treatments/medications.

### BACKGROUND OF THE INVENTION

There are a large number of chronic or long term health conditions that are challenging to treat. Diseases such as neuro-degenerative disorders, cancer, diabetes, IBD have shown to possess an elusive pharmaco-therapeutic target and are highly treatment-resistant.

Most pharmacological treatments/intervention/therapies have been shown to cause a wide variety of adverse and side effects. Many pharmacological treatments consist of chemicals that show extensive side effects, often related to interactions between the chemicals and other pharmacological targets. These other pharmacological targets can be expressed in other tissues or organs. For example, the endocannabinoid system is expressed in the central nervous system and digestive tract (CB1) in addition to the peripheral nervous system, immune system and digestive tract (CB2), thus the biological effect of various cannabinoids depends on the affinity to the different cannabinoid receptors. Additional mechanisms leading to side effects are variations in pharmacokinetics or drug-drug interactions. In the case of Parkinson's disease, for example, some of the side effects are related to the interaction of L-dopa (the therapeutic chemical) with receptors in the peripheral nervous system whereas, in the central nervous system (CNS), L-dopa is selectively metabolized to dopamine.

One solution for improving the efficacy of the therapeutics and limiting side effect is to selectively target the pharmacological composition to the tissue or organ affected by the disorder. Selective delivering of the therapeutic agent to a target tissue or organ can enable the use of a lower dosage, further lowering the chance of adverse effects.

There is an unmet need for a targeted therapeutic for the treatment of chronic and long term medical conditions.

### SUMMARY OF THE INVENTION

The present invention discloses a composition for tissue-specific and cell-specific delivery of active ingredients which may be active pharmaceutical ingredients (API) or active neutraceutical ingredients (ANI). The specific delivery is obtained by means of a specific nutraceutical ligand, thus enabling the delivery of medicaments and food supplements to specific sites, therefore providing a long-needed aid to subjects suffering from various ailments.

It is hence one object of the invention to disclose a composition for delivering an active pharmaceutical ingredient (API) to a targeted tissue or a targeted cell of a mammalian body, comprising a complex comprising of: at least one nanoparticle; at least one API; the API is contained by the nanoparticle; at least one nutraceutical ligand, the nutraceutical ligand is configured to specifically bind to the targeted tissue or the targeted cell; wherein the API is activated at the targeted tissue or at the targeted cell by the mediation of the nutraceutical ligand.

It is another object of the invention to disclose the composition as defined above, wherein the composition further comprises a linker that binds the at least one nutraceutical ligand to the at least one nanoparticle.

It is another object of the invention to disclose the composition as defined in any of the above, wherein the nutraceutical is a targeting nutraceutical.

It is another object of the invention to disclose the composition as defined in any of the above, wherein the nanoparticle is selected from the group consisting of a liposome, a micelle, a solid-lipid nanoparticle, a cyclodextrin nanoparticle, a dendrimer, a polymeric nanoparticle, a micro/nano-emulsion, and any combinations thereof.

It is another object of the invention to disclose the composition as defined in any of the above, wherein the micelle is a mono-layer or a bi-layer construction.

It is another object of the invention to disclose the composition as defined in any of the above, wherein the diameter of the nanoparticle is in the range of 10-250 nm.

It is another object of the invention to disclose the composition as defined in any of the above, wherein the API is bound to the nanoparticle via a chemical or a physical bond.

It is another object of the invention to disclose the composition as defined in any of the above, wherein the API is contained within the mono-layer or bi-layer, contained between layers of the bi-layer, or bound to an external layer of the mono-layer or bi-layer.

It is another object of the invention to disclose the composition as defined in any of the above, wherein the chemical or physical bond is selected from the group consisting of a covalent bond, a hydrogen bond, a wan der Waals bond, a hydrophobic bond, an electrostatic bond, a London bond, a ionic bond, a salt bridge, adsorption, and any combination thereof.

It is another object of the invention to disclose the composition as defined in any of the above, wherein the composition is adapted to release the API at the targeted tissue.

It is another object of the invention to disclose the composition as defined in any of the above, wherein the API is selected from a group consisting of a nutraceutical, a small molecule, a food supplement, a protein, a hormone, a peptide, and a fatty acid.

It is another object of the invention to disclose the composition as defined in any of the above, wherein the nutraceutical is an active nutraceutical ingredient (ANI).

It is another object of the invention to disclose the composition as defined in any of the above, wherein the ANI is selected from a group consisting of a neuro-active nutraceutical, an onco-active nutraceutical, an endo-active nutraceutical, a gastro-active nutraceutical, and any combination thereof.

It is another object of the invention to disclose the composition as defined in any of the above, wherein the ANI is curcumin.

It is another object of the invention to disclose the composition as defined in any of the above, wherein the API is generally recognized as safe (GRAS).

It is another object of the invention to disclose the composition as defined in any of the above, wherein the nutraceutical ligand is selected from a group consisting of a neuro-targeting compound, a tumor-targeting compound, a gastric targeting compound, an endo (pancreatic)-targeting compound, a cardiovascular targeting compound, a kidney targeting compound, an inflammation targeting compound, and any combination thereof.

It is another object of the invention to disclose the composition as defined in any of the above, wherein the neuro-targeting compound is selected from a group consisting of caffeine, theobromine (xantheose), paraxanthine, theophylline, 1,3,7-trimethyluric acid, guarana (Paullinia cupana, HBK), phenethylamine (PEA, chocolate), tryptamine (Acacia), and any combinations thereof.

It is another object of the invention to disclose the composition as defined in any of the above, wherein the neuro-targeting compound is selected from a group consisting of baicalein, wogonin, oroxylin A, and any combination thereof.

It is another object of the invention to disclose the composition as defined in any of the above, wherein the nutraceutical ligand is generally recognized as safe (GRAS).

It is another object of the invention to disclose the composition as defined in any of the above, wherein the composition is configured to achieve at least one of the following: a higher concentration of said API at the target tissue; a lower concentration of said API in non-targeted tissue; a higher activity of said API at the target tissue; an extended activity of said API at said target tissue; an increased delivery of the API to a target cell.

It is another object of the invention to disclose the composition as defined in any of the above, wherein the delivery mechanism of the API is characterized as being an active or a passive uptake mechanism.

It is another object of the invention to disclose the composition as defined in any of the above, wherein the uptake mechanism of the API is defined as the uptake of the at least one nanoparticle containing the at least one API, or of the at least one API released from the at least one nanoparticle.

It is another object of the invention to disclose the composition as defined in any of the above, wherein the uptake mechanism of the at least one nanoparticle containing the at least one API is endocytosis.

It is another object of the invention to disclose the composition as defined in any of the above, wherein the mode of the endocytosis is selected from the group consisting of receptor-mediated endocytosis, clathrin-mediated endocytosis, non-mediated endocytosis, caveolae, pinocytosis and phagocytosis.

It is another object of the invention to disclose the composition as defined in any of the above, wherein the active uptake mechanism is characterized as being facilitated by one of the group consisting of transporters, pumps and channels.

It is another object of the invention to disclose the composition as defined in any of the above, wherein the passive uptake mechanism is selected from the group consisting of diffusion, facilitated diffusion and filtration.

It is another object of the invention to disclose the composition as defined in any of the above, wherein the uptake is selected from the group consisting of secondary active transport, coupled transport or co-transport.

It is an object of the present invention to disclose a composition for delivering an API and/or ANI to the basal ganglia of a subject in need, comprising a plurality of nanoparticles covered with caffeine.

In some embodiments, the particles may be surface charged so complementary targeting agent can be bound by salt formation. For example, the lipid core is hydrogenated vegetable oil and the active agent within the core. The particles contain surfactants onto the surface. The surfactant may contain a positive or negative charge where by addition of caffeine the caffein binds to the surface by interacting with the charged surfactant onto the surface.

In some embodiments, there is provided a composition for delivering an active pharmaceutical ingredient (API) and/or active nutraceutical ingredient (ANI) to a basal ganglia of a subject in need thereof comprising:
a plurality of nanoparticles surface decorated with caffeine, wherein said nanoparticles comprising: at least one API or ANI, at least one emulsifier, at least one emulsifier stabilizer, at least one water miscible solvent, and at least one solid lipid matrix core;
and
a pharmaceutically or nutraceutically acceptable carrier.

In some embodiments, the emulsifier is PEG-40 hydrogenated castor oil (Cremophor RH40), polyoxyl 15 hydroxystearate, poloxamer 407 (Pluronic F127), lecithin (soy or sunflower, phosphatidylcholine content≥90%) and/or PEG-35 castor oil (Cremophor EL).

In some embodiments, the emulsion stabilizer is polysorbate 20, sorbitan monooleate, polysorbate 80 (Tween 80), polysorbate 60 (Tween 60), sorbitan monostearate (Span 60), and/or sorbitan monolaurate (Span 20).

In some embodiments, the water miscible solvent is propane-1,2-diol, polyethylene glycol 400 (PEG 400) and/or glycerol (glycerin), and/or 1,3-butanediol.

In some embodiments, the solid lipid matrix core is cocoa butter, cetyl palmitate, glyceryl behenate (Compritol 888 ATO), glyceryl palmitostearate (Precirol ATO 5) and/or beeswax.

In some embodiments, the nanoparticle is made of PEG-40 hydrogenated castor oil, polysorbate 20, sorbitan monooleate, polyoxyl 15 hydroxystearate, propane-1,2-diol and/or cocoa butter.

In some embodiments, the amount of PEG-40 hydrogenated castor oil is between 10-30% w/w, the amount of polysorbate 20 is between 10-30% w/w, the amount of sorbitan monooleate is between 10-30% w/w, the amount of polyoxyl 15 hydroxystearate is between 2-15% w/w, the amount of propane-1,2-diol is between 10-35% w/w, and the amount of cocoa butter is between 8-20% w/w.

In some embodiments, wherein the amount of PEG-40 hydrogenated castor oil is between 12-25% w/w, the amount of polysorbate 20 is between 12-25% w/w, the amount of sorbitan monooleate is between 12-25% w/w, the amount of polyoxyl 15 hydroxystearate is between 2-10% w/w, the amount of propane-1,2-diol is between 15-28% w/w, and the amount of cocoa butter is between 10-20% w/w.

In some embodiments, the amount of PEG-40 hydrogenated castor oil is between 15-20% w/w, the amount of polysorbate 20 is between 15-20% % w/w, the amount of sorbitan monooleate is between 15-20% w/w, the amount of polyoxyl 15 hydroxystearate is between 4-10% w/w, the amount of propane-1,2-diol is between 20-28% w/w, and the amount of cocoa butter is between 12-18% w/w.

In some embodiments, the amount of PEG-40 hydrogenated castor oil about 18% w/w, the amount of polysorbate 20 is about 18% w/w, the amount of sorbitan monooleate is about 18% w/w, the amount of polyoxyl 15 hydroxystearate is about 6% w/w, the amount of propane-1,2-diol is about 22% w/w, and the amount of cocoa butter is about 14% w/w.

In some embodiments, the API or the ANI is a lipophilic anti-inflammatory or antioxidant compound having a calculated Log P greater than 1.5 and a molecular weight of less than 900 Da.

In some embodiments, the API or the ANI is curcumin, coenzyme Q10 (ubiquinone), resveratrol, baicalein, alpha-tocopherol, astaxanthin, quercetin, honokiol, pterostilbene, fisetin, tanshinone IIA, carnosic acid, luteolin, ursolic acid, nobiletin, thymoquinone, apigenin, piperine, withaferin A, andrographolide, and any combination thereof.

In some embodiments, the API or the ANI is coenzyme Q10, resveratrol, baicalein, alpha-Tocopherol and/or astaxanthin.

In some embodiments, the API or the ANI is curcumin.

In some embodiments, the amount of the curcumin in the nanoparticle is between 1-8% w/w.

In some embodiments, the amount of the curcumin in the nanoparticle is about 4% w/w.

In some embodiments, there is provided a method of delivering API and/or ANI to the basal ganglia of a subject in need thereof comprising the step of intranasally administering the composition of the invention to the subject in need thereof, wherein the composition is a composition for delivering an active pharmaceutical ingredient (API) and/or active nutraceutical ingredient (ANI) to basal ganglia of a subject in need thereof comprising:
a plurality of nanoparticles surface decorated with caffeine, wherein said nanoparticles comprising: at least one API or ANI, at least one emulsifier, at least one emulsifier stabilizer, at least one water miscible solvent, and at least one solid lipid matrix core;
and
   a pharmaceutically or nutraceutically acceptable carrier.

In some embodiments, the API or ANI is curcumin.

In some embodiments, there is provided a method of manufacturing the nanoparticles in the composition of the invention comprising:
mixing PEG-40 hydrogenated castor oil, polysorbate 20, sorbitan monooleate, polyoxyl 15 hydroxystearate, propane-1,2-diol, cocoa butter and the API and/or the ANI to form a plurality of nanoparticles comprising the API and/or the ANI;
and
dispersing the plurality of nanoparticles in aqueous caffein dispersion.

In some embodiments, there is provided a method of treating Parkinson's Disease comprising the step of intranasally administering the composition of the invention to a subject in need thereof, wherein the composition is a composition for delivering an active pharmaceutical ingredient (API) and/or active nutraceutical ingredient (ANI) to a basal ganglia of a subject in need thereof comprising:
a plurality of nanoparticles surface decorated with caffeine, wherein said nanoparticles comprising: at least one API or ANI, at least one emulsifier, at least one emulsifier stabilizer, at least one water miscible solvent, and at least one solid lipid matrix core;
and a pharmaceutically or nutraceutically acceptable carrier.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are included to provide a further understanding of the invention and are incorporated in and constitute a part of this specification, illustrate embodiments of the invention and together with the description serve to explain the principles of the invention wherein:

FIG. 1 is a schematic representation of an embodiment of the composition.

FIG. 2 is a schematic representation of the binding activity of an embodiment of the composition.

FIG. 3 shows results of a toxicity experiment of an embodiment of the composition.

FIGS. 4a-e demonstrate results of API delivery experiment of an embodiment of the composition.

FIGS. 5a-b demonstrate results of API delivery experiment of another embodiment of the composition.

FIGS. 6a-c demonstrate imaging of API delivered by an embodiment of the composition in target cells.

FIGS. 7a-c demonstrate curcumin concentrations in plasma (panel 7A, ng/mL), striatum/basal ganglia (panel 7B, ng/g), and frontal cortex (panel 7C, ng/g) following intranasal administration of) Caffeine-SLN-Curcumin in Wistar rats (n=4 per group per time point). Caffeine-SLN-Curcumin demonstrated selective and superior accumulation of curcumin in the basal ganglia and frontal cortex at t=60 minutes.

### DETAILED DESCRIPTION OF THE EMBODIMENTS OF THE INVENTION

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide compositions and methods.

It is core objective the present invention to disclose and provide a platform for the targeted delivery of an active pharmaceutical ingredient (API) or nutraceutical ingredient (ANI) guided to the targeted mammalian cell, tissue or organ by a nutraceutical ligand. The platform would provide a nanoparticle having a shell decorated with at least one nutraceutical targeting compound or nutraceutical ligand and a core comprising at least one active pharmaceutical ingredient or nutraceutical compound. The nutraceutical ligand binds to specific receptors on the mammalian cell, tissue or organ.

In this application, the term “nanoparticle” (NP) relates to a particle between 1 and 250 nanometers (nm) in diameter, having a core surrounded by an interfacial layer (the shell). In some embodiments, the nanoparticle is selected from a liposome, a micelle, a solid-lipid nanoparticle, a cyclodextrin nanoparticle, a dendrimer, a polymeric nanoparticle, a micro/nano-emulsion and any combinations thereof.

In some embodiments, the NP is a vessel or a liposome, comprising a lipid bilayer or a single layer (a multilamellar liposome or a unilamellar liposome).

In an embodiment of the application, the outer surface of the nanoparticle (the shell) is decorated, by at least one targeting compound (a nutraceutical ligand), that can bind to the target. The target could be a tissue, organ or specific cell.

The term ‘Nutraceutical’ is defined as any substance that is a food or a part of the food and provides medical or health benefits, including the prevention and treatment of disease. These nutraceuticals including the isolated nutrients, dietary supplements (minerals, vitamins, amino acids, fatty acids, saccharides, probiotics, prebiotics, and herbs) and phytochemicals promised to have beneficial health effects besides the traditional nutrition values found in food. Nutraceuticals are commonly derived from food sources, plant matter, fungus, bacteria or microbes derived from a natural source, such as a plant or bacteria. The nutraceutical could be defined as an alkaloid, a phenol (or a polyphenol), a peptide, a saccharide, a terpene, phenolic, glycoside a fatty acid, a flavonoid, a phytosterol, and as tannins. The nutraceutical can serve as a primary or a secondary metabolite.

One of the major benefits of nutraceuticals over pharmaceuticals include a much faster “to market” period, lesser or no adverse effects, economically affordable, easily available, multiple therapeutic effects, and enables long term consumption without toxicity or titration concerns.

The decoration is achieved by any type of modification of the shell (chemically bonding to the shell, chemically coordinating with the shell, forming a part of the shell, polymerized into the shell, and so forth) or by modifying a nutraceutical ligand so as to bind to the shell. In some embodiments, the complex comprises a linker that binds to the nutraceutical ligand and to the NP. In a prominent non limiting example of the present invention, the nutraceutical ligand is caffeine, and the API is an ANI, namely curcumin.

The invention further provides a nanoparticle-based composition comprising a nanoparticle-shell decorated with at least one neuro-targeting nutraceutical and a core comprising at least one neuro-active nutraceutical, for use in the treatment of at least one neurodegenerative disease, neoplastic disease, digestive disease, endocrine disorders, kidney disorders, cardiovascular diseases and any symptom thereof.

An active pharmaceutical ingredient (API) is defined as “any substance or combination of substances used in a finished pharmaceutical product (FPP), intended to furnish pharmacological activity or to otherwise have direct effect in the diagnosis, cure, mitigation, treatment or prevention of disease, or to have direct effect in restoring, correcting or modifying physiological functions in human beings” (WHO, 2011). The API can be classified according to its structure (such as antibodies), its therapeutic target (such as analgesics) or its biological effect (such as Non-steroidal anti-inflammatory drug—NSAID). The API can be a small molecule (such as NSAID) or a macromolecule (such as antibodies). The API can be from a natural source (such as a natural product) or produced in a semi-synthetic or synthetic manor.

In some embodiments, the API is delivered to the target tissue or cell through an active or a passive uptake mechanism. In some embodiments, the uptake mechanism is of the whole API-contained NP, such as endocytosis. The endocytosis can be mediated, such as receptor-mediated endocytosis (also known as clathrin-mediated endocytosis), or non-mediated (such as caveolae, pinocytosis, and phagocytosis). In some embodiments, the API is released by the NP into the extracellular matrix and the API uptake is of a released (un-bound) API. The uptake can be an active mechanism (such as transporters, pumps, and channels) or passive (such as diffusion, facilitated diffusion and filtration). In some embodiments, the transport is secondary active transport (known as coupled transport or cotransport).

In some embodiments, the API is a nutraceutical. This API is referred to as active nutraceutical ingredient (ANI).

As used herein, the term “SLN” or “solid lipid nanoparticle” refers to a colloidal nanocarrier comprising a solid lipid matrix core stabilized by one or more surfactants and co-surfactants, with mean particle diameter in the range of 50-500 nm In some embodiments, the diameter size is between 75-400 nm. In some embodiments, the diameter size is between 100-300 nm. In some embodiments, the diameter size is between 150-250 nm. In some embodiments, the diameter size is about 200 nm.

In some embodiments, composition of the invention comprising nanoparticles that may be surface charged so complementary targeting agent can be bound by salt formation. For example, the lipid core is hydrogenated vegetable oil and the active agent within the core. The nanoparticles contain surfactants onto the surface. The surfactant may contain a positive or negative charge where by addition of caffeine, the caffeine binds to the surface by interacting with the charged surfactant onto the surface.

As used herein, the term ‘basal ganglia” are a cluster of subcortical nuclei deep to cerebral hemispheres. The largest component of the basal ganglia is the corpus striatum, which contains the caudate and lenticular nuclei (the putamen, globus pallidus externus, and internus), the subthalamic nucleus (STN), and the substantia nigra (SN).

As used herein, the term “Pro-Nano-Lipid pre-concentrate” or “PNL pre-concentrate” refers to a thermodynamically stable, isotropic liquid mixture of lipid, surfactants, co-solvent, and active ingredient that spontaneously disperses into SLN upon contact with aqueous media at or above the melting point of the lipid matrix.

As used herein, the term “Caffeine-SLN” refers to SLN prepared according to the methods of the specification, whose surface has been covered with caffeine by the aqueous dispersion method described herein.

As used herein, the term “Uncoated SLN” refers to SLN prepared by the same method but dispersed into caffeine-free aqueous medium, serving as a comparator formulation in the in vivo study.

As used herein, the term “Mean particle size” refers to the Z-average hydrodynamic diameter as measured by dynamic light scattering (DLS). In some embodiments, the experiments were conducted at 25 °C for the stability tests or at 40 °C for freshly prepared dispersions.

As used herein, the term “PDI” refers to the polydispersity index as reported by DLS, ranging from 0 (monodisperse) to 1 (highly polydisperse).

As used herein, the term “Zeta potential” refers to the electrokinetic potential of the particle surface measured by electrophoretic light scattering.

As used herein, the term “Caffeine binding efficiency” refers to the percentage of added caffeine that remains associated with isolated SLN following centrifugation-based separation of free (unbound) caffeine from particle-bound caffeine.

As used herein, the term “DDW” refers to double-distilled water.

As used herein, the term “MilliQ water” refers to ultrapure water with resistivity≥18.2 MΩ·cm at 25 °C (acceptable substitute for DDW throughout).

Examples 7 and 8 detail the preparation of Caffeine-coated Curcumin-loaded solid lipid nanoparticles (“Caffeine-SLN-Curcumin”).

Curcumin (1,7-bis(4-hydroxy-3-methoxyphenyl)hepta-1,6-diene-3,5-dione; CAS 458-37-7) is a hydrophobic polyphenol derived from the dried rhizomes of Curcuma longa L. Despite a well-documented pharmacological profile that includes anti-inflammatory, antioxidant, and neuroprotective activities, curcumin's clinical utility has been severely constrained by its extremely low aqueous solubility (<0.1 μg/mL at physiological pH), rapid first-pass metabolism, and negligible blood-brain barrier (BBB) permeability following oral administration.

Caffeine (1,3,7-trimethylxanthine; CAS 58-08-2) is a methylxanthine that is known to interact with adenosine receptors broadly distributed throughout the striatum and basal ganglia. The inventors found that caffeine, is adsorbed spontaneously onto the negatively charged surface of a curcumin-loaded SLN through non-covalent interactions, without requiring covalent conjugation chemistry. The resulting caffeine-decorated particle surface surprisingly led to curcumin accumulation in striatal tissue relative to uncoated particles and free curcumin as can be seen in Example 10. Further, the intranasal administration of Caffeine-SLN-Curcumin to rats resulted in high concentrations of curcumin in the basal ganglia of the tested rats, as demonstrated in Example 10.

In some embodiments, the intranasal (IN) route offers a clinically attractive alternative: axonal and olfactory pathways connecting the nasal mucosa to the central nervous system (CNS) allow direct brain deposition, bypassing both hepatic first-pass metabolism and the BBB. However, successful in delivery of hydrophobic actives requires a nanocarrier system capable of encapsulating curcumin at pharmacologically meaningful payload levels, generating particles that interact productively with the olfactory epithelium, and surviving transit to the CNS without premature drug release.

In some embodiments, there is provided a composition for delivering an active pharmaceutical ingredient (API) and/or active nutraceutical ingredient (ANI) to a basal ganglia of a subject in need thereof comprising: a plurality of nanoparticles covered with caffeine wherein the nanoparticles comprising: at least one API or ANI, at least one emulsifier, at least one emulsifier stabilizer, at least one water miscible solvent and at least one solid lipid matrix core; and a pharmaceutically or nutraceutically acceptable carrier.

In some embodiments, the emulsifier is PEG-40 hydrogenated castor oil, polyoxyl 15 hydroxystearate, poloxamer 407 (Pluronic F127), lecithin (soy or sunflower, phosphatidylcholine content≥90%), and/or PEG-35 castor oil (Cremophor EL).

In some embodiments, the emulsion stabilizer is polysorbate 20, sorbitan monooleate, polysorbate 80 (Tween 80), polysorbate 60 (Tween 60), sorbitan monostearate (Span 60), and/or sorbitan monolaurate (Span 20)

In some embodiments, the water miscible solvent is propane-1,2-diol, polyethylene glycol 400 (PEG 400), glycerol (glycerin), and/or 1,3-butanediol. In some embodiments, the Solid lipid matrix core is cocoa butter, glyceryl monostearate (GMS), cetyl palmitate, glyceryl behenate (Compritol 888 ATO), glyceryl palmitostearate (Precirol ATO 5) and/or beeswax.

In some embodiments, the nanoparticle is made of PEG-40 hydrogenated castor oil, polysorbate 20, sorbitan monooleate, polyoxyl 15 hydroxystearate, propane-1,2-diol and cocoa butter.

In some embodiments, the amount of PEG-40 hydrogenated castor oil is between 10-30% w/w, the amount of polysorbate 20 is between 10-30% w/w, the amount of sorbitan monooleate is between 10-30% w/w, the amount of polyoxyl 15 hydroxystearate is between 2-15% w/w, the amount of propane-1,2-diol is between 10-35% w/w and the amount of cocoa butter is between 8-20% w/w.

In some embodiments, the amount of PEG-40 hydrogenated castor oil is between 12-25% w/w, the amount of polysorbate 20 is between 12-25% w/w, the amount of sorbitan monooleate is between 12-25% w/w, the amount of polyoxyl 15 hydroxystearate is between 2-10% w/w, the amount of propane-1,2-diol is between 15-28% w/w, and the amount of cocoa butter is between 10-20% w/w.

In some embodiments, the amount of PEG-40 hydrogenated castor oil is between 15-20% w/w, the amount of polysorbate 20 is between 15-20% % w/w, the amount of sorbitan monooleate is between 15-20% w/w, the amount of polyoxyl 15 hydroxystearate is between 4-10% w/w, the amount of propane-1,2-diol is between 20-28% w/w and the amount of cocoa butter is between 12-18% w/w.

In some embodiments, the amount of PEG-40 hydrogenated castor oil about 18% w/w, the amount of polysorbate 20 is about 18% w/w, the amount of sorbitan monooleate is about 18% w/w, the amount of polyoxyl 15 hydroxystearate is about 6% w/w, the amount of propane-1,2-diol is about 22% w/w and the amount of cocoa butter is about 14% w/w.

In some embodiments, the API or the ANI is a lipophilic anti-inflammatory or antioxidant compound having a calculated Log P greater than 1.5 and a molecular weight of less than 900 Da.

In some embodiments, he composition of claim 1, wherein the API or the ANI is curcumin, coenzyme Q10 (ubiquinone), resveratrol, baicalein, alpha-tocopherol, astaxanthin, quercetin, honokiol, pterostilbene, fisetin, tanshinone IIA, carnosic acid, luteolin, ursolic acid, nobiletin, thymoquinone, apigenin, piperine, withaferin A, andrographolide, and any combination thereof.

In some embodiments, the API or the ANI is coenzyme Q10, resveratrol, baicalein, alpha-Tocopherol and/or astaxanthin.

In some embodiments, the API or the ANI is curcumin.

In some embodiments, the amount of the curcumin in the nanoparticle is between 1-8% w/w.

In some embodiments, the amount of the curcumin in the nanoparticle is about 4% w/w.

In some embodiments, the amount of the curcumin in the nanoparticle is less than 4%, 3.5%, 3%, 2.5%, 2%, 1.5%, 1% w/w or less.

In some embodiments, there is provided a method of delivering API and/or ANI to the basal ganglia of a subject in need thereof comprising the step of intranasally administering the composition of the invention to the subject in need thereof.

In some embodiments, the composition comprises nanoparticles comprising curcumin that are coated by caffeine.

In some embodiments, the composition is administered internasally and the curcumin accumulates in the basal ganglia of the subject in need thereof.

In some embodiments, there is provided a method of manufacturing the nanoparticles in the composition of the invention comprising: mixing PEG-40 hydrogenated castor oil, polysorbate 20, sorbitan monooleate, polyoxyl 15 hydroxystearate, propane-1,2-diol, cocoa butter and the API and/or the ANI to form a plurality of nanoparticles comprising the API and/or the ANI; and dispersing the plurality of nanoparticles in aqueous caffeine dispersion.

In some embodiments, a vial comprising the mixed plurality of nanoparticles comprising the API and/or the ANI is placed in an oil bath or temperature-controlled water bath set to 37-50 °C. The mixture in some embodiments, is stirred continuously using a magnetic stir bar at moderate speed for a period of for example, 1-3 hours, until complete homogeneity is achieved.

In some embodiments, the resulting pre-concentrate (hereinafter “SLN pre-concentrate” or “PNL pre-concentrate”) is a thermodynamically stable, single-phase mixture that disperses spontaneously in aqueous media upon contact. The pre-concentrate is stable when stored at room temperature (20-25 °C) in the dark, protected from moisture.

In some embodiments, cocoa butter is used as a solid lipid matrix for the nanoparticles of the composition of the invention. Earlier developmental batches employed sesame oil as an alternative lipid phase; however, cocoa butter demonstrated superior nanoparticle stability and reproducibility

In some embodiments, prior to dispersion, the SLN pre-concentrate is warmed to a temperatures of between 25-40 °C, optionally 37 °C for a minimum of 5, 10.15 minutes and is mixed by shaking or inversion to ensure complete homogeneity.

Uncoated SLN is prepared in some embodiments, by adding the pre-concentrate to water that may be pre-heated to 25-40 °C, optionally 37 °C, in for example a ration of 1:10 of pre-concentrate to aqueous phase (10% v/v pre-concentrate). In some embodiments, the ratio of pre-concentrate to water is more than 1:5. In some embodiments, the ratio of pre-concentrate to water is more than 1:10. In some embodiments, the ratio of pre-concentrate to water is more than 1:15. In some embodiments, the ratio of pre-concentrate to water is more than 1:20. In some embodiments, the ratio of pre-concentrate to water is more than 1:25. Immediately upon addition, the mixture is vortexed vigorously for about 5, 10, 15, 20, 30, 6-, 75, 90, 120 or more seconds until a homogeneous, optically clear to slightly opalescent dispersion was obtained. This step is referred to as the “dispersion step.”

In some embodiments, spontaneous nanoparticle formation occurs as the amphiphilic surfactant-rich pre-concentrate contacts the aqueous medium, driving self-assembly of lipid nanoparticles with the curcumin molecules sequestered within the hydrophobic cocoa butter matrix. No high-pressure homogenization, sonication, or other high-energy input is required; the process is driven entirely by the thermodynamic affinity of the formulation components for ordered interfacial assembly.

The ratio of pre-concentrate to aqueous phase may be varied within the range of 1:5 to 1:20 v/v without loss of nanoparticle integrity. In some embodiments, a ratio of about 1:10 v/v is preferred for its optimal balance of curcumin concentration and particle size uniformity.

In some embodiments, Caffeine-decorated SLN (Caffeine-SLN-Curcumin) are prepared by replacing the DDW dispersion medium with an aqueous caffeine solution.

In some embodiments, caffeine is dissolved in DDW or MilliQ water. In some embodiments, concentration of the caffeine is about 1.0 mg/mL (0.10% w/v). In some embodiments, the caffeine solution is heated to for example, to about 37 °C, to assist dissolution. In some embodiments, the caffeine mixture is vortexed immediately, for example, for 15-120 seconds.

In some embodiments, caffeine adsorption onto the SLN surface occurs via non-covalent interactions between the caffeine molecule and the hydrophobic lipid-surfactant interface of the negatively charged SLN. In some embodiments, the anionic character of the freshly formed SLN surface facilitates electrostatic and hydrophobic association with caffeine. No covalent bond formation is required and no coupling reagents or reactive chemistries are involved.

In some embodiments, the effective caffeine concentration at the particle surface corresponds to about 1-20% w/w relative to the total lipid phase (curcumin plus cocoa butter combined).

In some embodiments, the effective caffeine concentration at the particle surface corresponds to about 1-10% w/w relative to the total lipid phase (curcumin plus cocoa butter combined).

In some embodiments, the effective caffeine concentration at the particle surface corresponds to approximately 5% w/w relative to the total lipid phase (curcumin plus cocoa butter combined).

In some embodiments, the freshly prepared SLN dispersion (both uncoated and caffeine-coated) is subjected to filtration.

In some embodiments, the filtration is at 30-45 °C (rather than at room temperature because performing filtration at room temperature leads to lipid solidification at the filter membrane with concomitant curcumin retention.

In some embodiments, the filtration is at 40 °C (rather than at room temperature because performing filtration at room temperature leads to lipid solidification at the filter membrane with concomitant curcumin retention. At 40 °C, the lipid matrix remains sufficiently fluid to pass through the membrane.

In some embodiments, the filtration step substantially improves the polydispersity index (PDI) of the dispersion, reducing it from approximately 0.31 in unfiltered samples to approximately 0.23 in filtered samples, without measurable loss of curcumin content. As can be seen in the examples, curcumin retention was confirmed by UV-Vis absorbance at the curcumin λmax of 426 nm, measured after complete dissolution of the nanoparticles in absolute ethanol; filtered and unfiltered samples produced equivalent absorbance values at this wavelength.

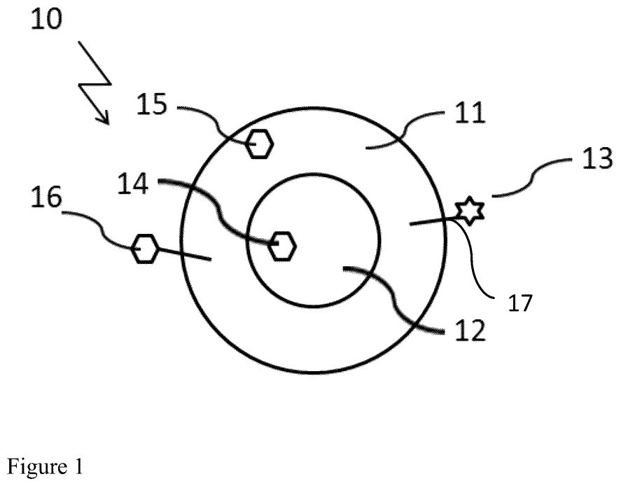

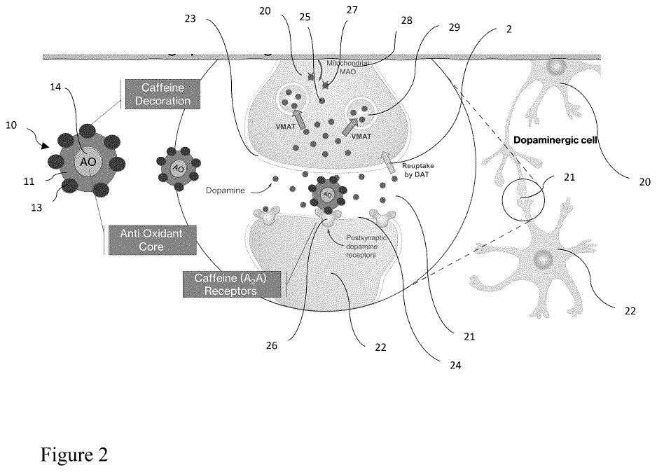

Reference is made to FIG. 1, showing an embodiment of the composition, comprising a nanoparticle 10, the nanoparticle 10 comprising a bilayer liposome 11 with an internal volume 12. The targeting compound 13 is bound to the outer shell via a linker 17, while an API is either contained within the internal volume 14, within the bilayer 15 or bound to the external shell 16. Reference is made to FIG. 2, showing schematic representation of the binding activity of an embodiment of the composition. The composition comprises a nanoparticle 10 decorated with caffein ligands 13 on the outer surface of the bi-layer 11 of the nanoparticle 10. The nanoparticle 10 contains an anti-oxidative neuro-active API 14. The nanoparticle 10 binds the target cell 22 thereof, via the caffein ligand 13. This target cell 22 is a postsynaptic cell comprising postsynaptic dopamine receptors 26 on the postsynaptic membrane 24 thereof. Dopamine molecules 25 are released by a presynaptic cell 20 via vesicular monoamine transporters (VMAT) 29, into a synapse 21. The excess molecules 25 are later reuptaken from the synapse 21 by a dopamine transporter (DAT) 2, back into the presynaptic cell 20, where they are catabolized 27 by mitochondrial monoamine oxidase (MAO) 28. In other embodiments, nanoparticles can be decorated by DAT-binding ligands.

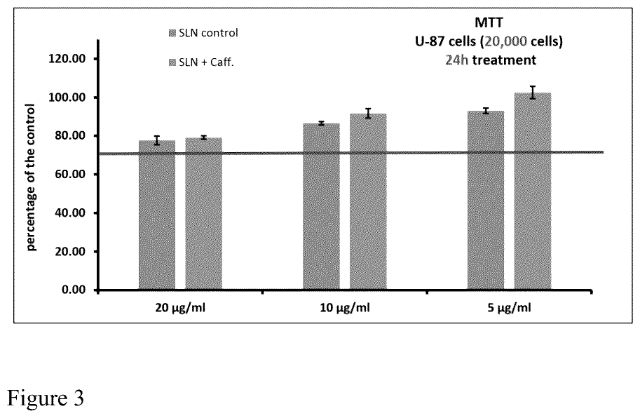

Reference is made to FIG. 3, showing results of an in-vitro toxicity experiment of an embodiment of the composition. Human Astrocytes (U-87 MG cell line) were exposed to solid lipid nanoparticles (SLNs) 10 decorated by caffein ligands 13, and containing the ANI curcumin 14. Cell viability was determined using 3-(4,5-dimethylthiazol-2-yl)-2,5-diphenyltetrazolium bromide (MTT) assays. After overnight stabilization, cells were exposed to varying concentrations of the SLNs 10. After 24 hours, the media was removed, and all cells were incubated with cerum-free media containing MTT for 3-4 hours in the incubator. The MTT purple crystals formed by the viable cells were diluted using isopropanol containing 0.04 mol/L HCl. Optical density was measured at 570 nm. Data is shown as percentage of untreated cells (control). Data are mean±SE (n=4). No toxic effect was observed following the SLN 10 treatments with the above mentioned concentrations. The cell viability of the treated cells was even higher than of the control cells.

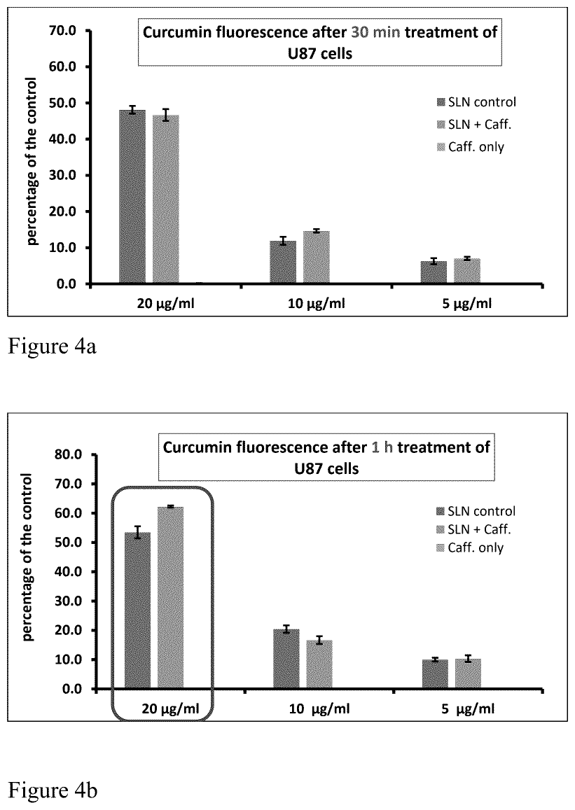

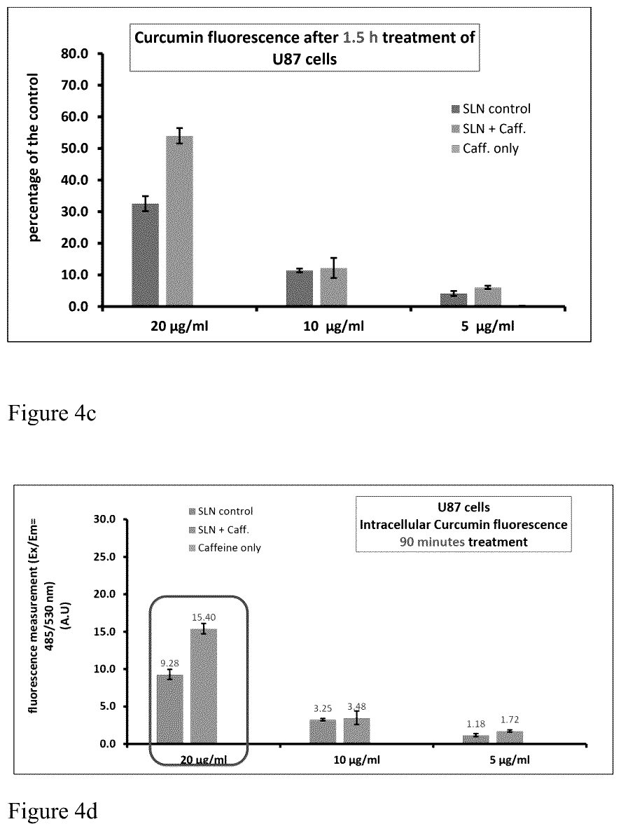

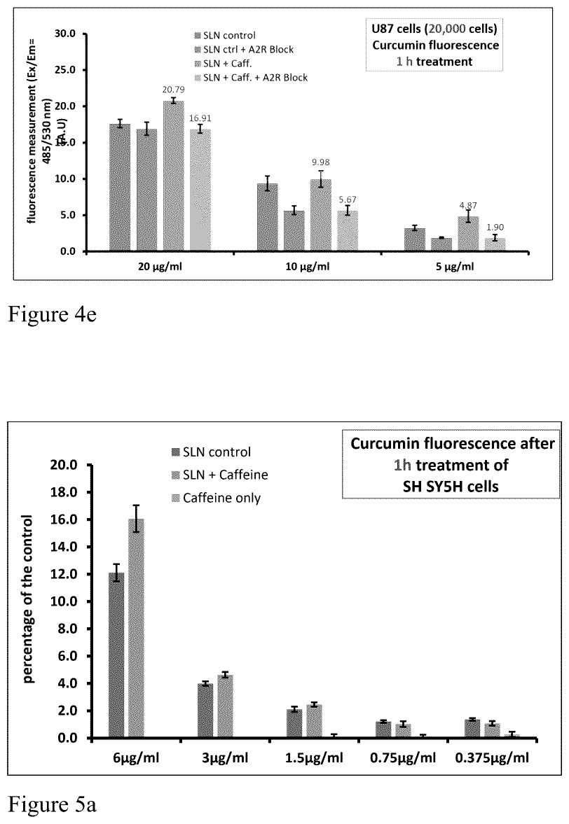

Reference is made to FIGS. 4a-e, showing results of an in-vitro curcumin delivery experiment of an embodiment of the composition. Human Astrocytes (U-87 MG cell line) were exposed to solid lipid nanoparticles (SLNs) 10 decorated by caffein ligands 13, and containing the ANI curcumin 14. FIGS. 4a-c depict different time points of the same experiment. Cells were seeded and grown for 24 h before treatment. Each time point was measured in a different plate. Media was replaced with fresh media containing the different treatments. After the treatment duration, cells were washed using PBS X1 and lysed using RIPA buffer. After 5 min of agitation, fluorescence was measured using curcumin specific wavelength of curcumin 14 (Ex/Em=485/530 nm). The results are shown as percentage of a curcumin sample (served as control). Data are mean±SE (n=4). As shown in FIG. 4a, no significant difference between SLNs 10 decorated with caffein 13 and ligand-less SLNs 10 were spotted after 0.5 hour. However, as depicted in FIGS. 4a-d, after 1 h (FIG. 4b) and 1.5 h (FIGS. 4c-d) of treatment of Human Astrocytes (U-87 MG cell line), a significant increase in the curcumin fluorescence was observed following treatment with Caffeine-SLN-Curcumin (Group C), as compared to Uncoated SLN-Curcumin (Group B) and the free curcumin control (Group A). No significant difference between Caffeine-SLN-Curcumin and Uncoated SLN-Curcumin was observed at t=0.5 hours. The caffeine coating of the SLN augments intracellular curcumin content in a time-dependent manner. This indicates that the coating of the sample with caffeine 13 augments the intracellular content of curcumin 14 at 1.5 hours. An experiment similar to FIG. 4c is depicted on FIG. 4d. In both experiments, measurement were taken after 1.5 hours of treatment. The only difference is the absolute amount of curcumin 14 in the cells. However, the relations between the results of the different treatments are similar in FIG. 4c and FIG. 4d. FIG. 4e depicts results of an in-vitro curcumin delivery experiment of an embodiment of the composition, which is similar to the experiment depicted in FIG. 4d, with an addition of another control variable: A2R receptor blocker, which was used since caffeine 13 is an antagonist to A2R receptors. The results indicates that indeed the blocking of A2R receptors with an antagonist, reduces the curcumin content to the same levels as the SLN control (in the concentration of 20 mg/ml).

Reference is made to FIGS. 5a-b, showing results of an in-vitro curcumin delivery experiment of another embodiment of the composition. Human Neuroblastoma cells (SH Sy5Y cells cell line) were exposed to solid lipid nanoparticles (SLNs) 10 decorated by caffein ligands 13, and containing the ANI curcumin 14. FIGS. 5a-b depict different time points of the same experiment. As depicted in FIG. 5a, after 1 hour, a significant increase in the curcumin 14 fluorescence was observed following SLN 10+Caffeine 13 (Caffeine-SLN-curcumin) sample, as compared to the SLN 10 control sample and to curcumin 14 sample. However, as depicted in FIG. 5b, after 3.5 hours of treatment, the fluorescence of the SLN 10 control sample and the curcumin 14 sample remained at the same levels, as opposed to the SLN 10+Caffeine 13 (Caffeine-SLN-curcumin) sample, which diminished considerably. This effect might be due to intra-cellular metabolism of the curcumin present in the SLN 10+Caffeine 13 (Caffeine-SLN-curcumin) sample.

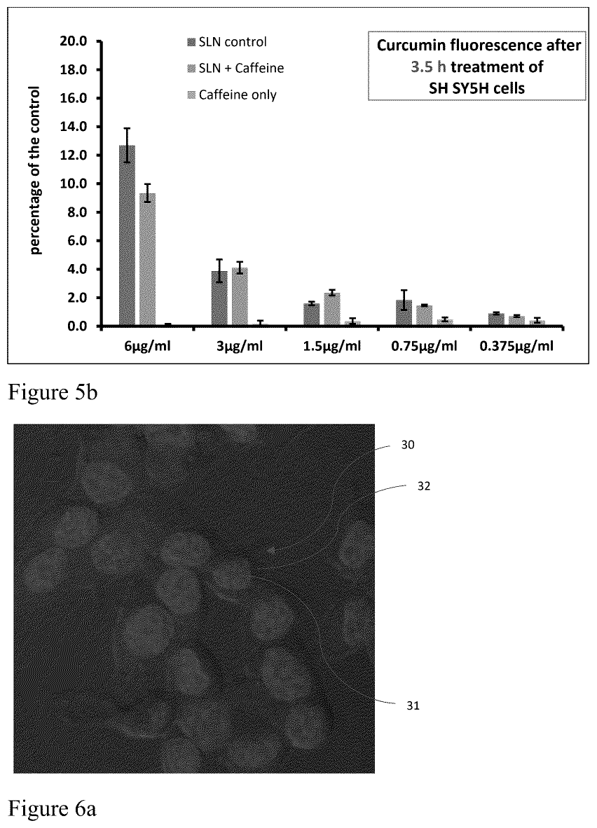

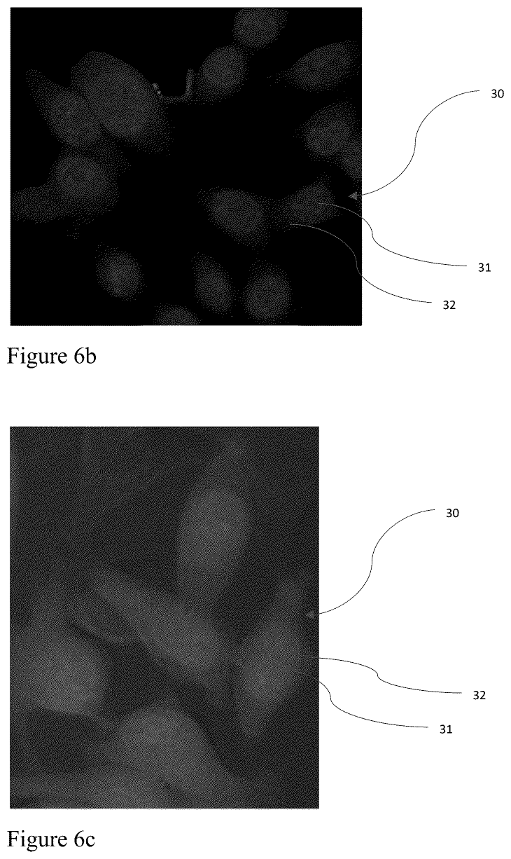

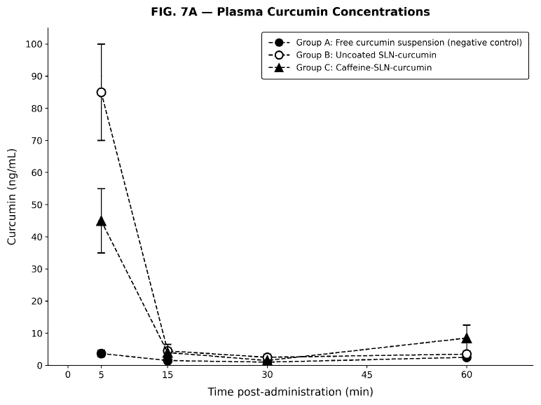

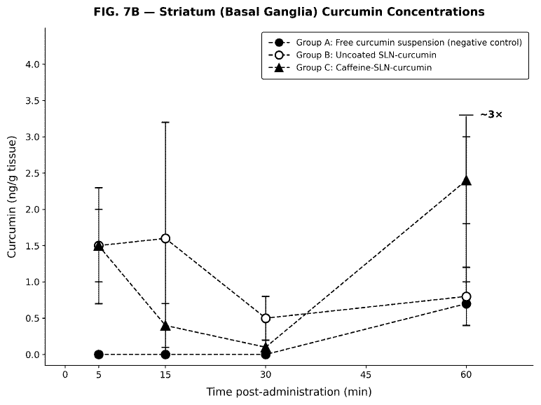

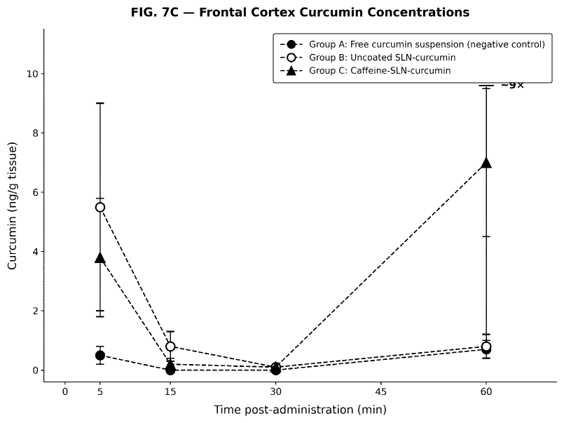

Reference is made to FIGS. 6a-c, showing fluorescence imaging of Human Astrocytes (U-87 MG cell line) exposed to solid lipid nanoparticles (SLNs) 10, the SLNs 10 being decorated by caffein ligands 13 and containing the ANI curcumin 14. The cells 30 were seeded in a 24-well plate at a density of 8×10\hat{ }4 cells/well and were incubated for overnight stabilization. After the treatment, the cells 30 were washed with PBS X1 and fixate using 3% paraformaldehyde for twenty minutes. After second washing with PBS, the cellular nucleus 31 was stained using Hoechst 33342. The analysis was performed using Nikon's A1R+ confocal microscope in The Hebrew University of Jerusalem Interdepartmental Equipment Facility. In this imaging assay, the green fluorescence in the pictures indicates the presence of curcumin 32 in the cytoplasm, while the blue color indicates the nuclei 31 of the cells. FIG. 6a depicts untreated control cells, FIG. 6b depicts cells exposed linandless control SLNs 10 for 1.5 hours, and FIG. 6c depicts cells exposed to SLNs 10 decorated by caffeine ligands 13 for 1.5 hours. A significantly higher curcumin 32 staining can be observed in the SLN 10+Caffeine 13 (Caffeine-SLN-curcumin) treatment at 1.5 hours, compared to SLN 10 control and untreated cells 30. In untreated cells 30, green color is not detectable. In SLN 10 control cells 30, certain amount of green color is detectable around the nuclei 31 of the cells 30. In caffeine-decorated cells 30, the green color is so strong it even interferes with the blue color of the nucleus 31. Numerical analysis of the fluorescence images will be provided later.

Targeting activity: In this application, the term “targeting nutraceutical” refers to a compound that is derived from a natural source and has been clinically substantiated to contribute in delivering a nanoparticle of the invention in a manner that increases the local concentration of the nanoparticle in specific organ or tissue, such as in the central nervous system (CNS) or parts of it, relative to other parts of the subject body. The targeting nutraceutical can also be referred to as a bio-carrier, as its ability to ‘carry’ the nanoparticle conjugate to the target organ, tissue or cell. The goal of a targeted delivery system of the invention is to provide a targeted, localized, extended/long-effect interaction with the target. The targeting nutraceutical can be targeted to various systems and disorders, such as the central or peripheral nervous system (such as Parkinson's disorder, Alzheimer's disease or multiple sclerosis), neoplastic growths (tumor), endocrine system (such as diabetics) or the gastrointestinal system (such as gastric ulcers or IDB).

The advantages to the targeted delivery relate to the localized concentration of the medication in the tissues of interest while the reduction in the relative concentration of the medication in the remaining tissues. This strategy can lead to various beneficial effects:
a. Lower doses of active compounds;
b. A reduction in the frequency of the dosages taken by the subject;
c. A more consistent pharmacological effect;
d. A reduction of harmful side-effects by reducing and controlling changes in circulating levels of the nanoparticle of the invention.

In some embodiments, the targeting compound is a neuro-targeting nutraceutical:

In some embodiments, the neuro-targeting nutraceutical is selected from caffeine and its four dimethylxanthines-metabolites, including, Theobromine (xantheose), Paraxanthine, Theophylline, and 1,3,7-Trimethyluric acid, guarana (Paullinia cupana, HBK), Phenethylamine (PEA, chocolate), Tryptamine (Acacia) and any combinations thereof. Caffeine is an adenosine receptor antagonist (A_{2A}R), which is expressed in PD target sites (Svenningsson et al. 1999, Madreira et al., 2017; Rivera-Oliver et al., 2014). In some embodiments, the targeting nutraceutical is selected from theobromine (xantheose), a bitter alkaloid of the cacao plant, and its alkaloid analogs.

In some embodiments, the targeting compound is another group of neuro-targeting nutraceuticals, specifically targeting cell bodies of dopamine neurons: baicalein, wogonin, and oroxylin A. These neuro-targeting nutraceutical bind the same receptors as cocaine, and are therefore likely to be useful in treatment of substance addictions.

Since dopamine receptors are mostly post-synaptic, DATs (dopamine transporters), are selected for targeting. DATs are membrane-spanning transporters that pump dopamine out of the synaptic cleft back into the cytosol. Each DAT is composed of twelve transmembrane domains (TMD) with a large extracellular loop between the third and fourth TMDs [Ben-Jonathan, Nira. Dopamine. CRC Press. Kindle Edition].

Unlike amino acid transporters, which are found on both neurons and glia, the expression of DAT is restricted to dopaminergic neurons, making DAT a well-accepted experimental and pathological marker for these neurons. At the cellular level, DAT is found in both dendrites and cell bodies of neurons in the substantia nigra and ventral tegmental areas.

In view of the above, the neuro-targeting nutraceutical, natural ligands for DAT are proposed as baicalein, wogonin, and oroxylin A, which are main active flavones in the medicinal herbs Scutellaria baicalensis and Scutellaria lateriflora. [Ji, Yeongseon, et al. “Neuroprotective effects of baicalein, wogonin, and oroxylin A on amyloid beta-induced toxicity via NF-κB/MAPK pathway modulation.” Molecules 25.21 (2020): 5087)].

In some embodiments, the targeting nutraceutical is an onco-targeting nutraceutical:

In some embodiments, the onco-targeting nutraceutical is selected from saccharides and sugar substitutes. In some embodiments, the targeting nutraceutical is a modified sugar, such as sucralose (chlorinated sucrose). In some embodiments, targeting nutraceutical is sugar alcohol such as sorbitol, xylitol, erythritol, mannitol, maltitol, lactitol, erythritol. In some embodiments the sugar substitute is Saccharin. The modified sugar or sugar alcohol can demonstrate slower metabolism and higher accumulation in the extravascular extracellular space (EES) of tumors.

In some embodiments, the targeting nutraceutical is an endocrine-targeting nutraceutical:

In some embodiments, the endocrine-targeting nutraceutical is selected from a group of insulin analogs and insulin receptor agonists/antagonists. The Insulin receptor is a transmembrane tyrosine kinase receptor that binds to insulin and insulin growth factors (I and II). In some embodiments, the insulin analog is Momordica charantia insulin receptor-binding protein or a peptide segment of the protein. In some embodiments, the insulin receptor can be activated by Biotin, Magnesium, Chromium, Gymnema s., Camellia s. and Vaccinium m. [see Covolo et al. (2017) BMC Public Health, 13, 777].

In some embodiments, the targeting nutraceutical is a gastro-targeting nutraceutical:

In some embodiments, the NP comprises a nutraceutical-based coating that lowers the biodiversity of the NP and increases the delivery of the NP to the intestine. In some embodiments, the coating lowers the solubility of the NP. The coating can be lipid or saccharide based. In some embodiments, the coating increases the resistance of the NP to the conditions in the gastrointestinal tract, such as the stomach or and the intestine.

In some embodiments, the gastro-targeting nutraceutical is a long-chain fatty acid (LCFA), such as oleic acid, linoleic acid, and linolenic acid, that binds to the Intestinal-fatty acid-binding protein (I-FABP) in small intestinal epithelial cell lines.

In some embodiments, the NP comprises a kidney or heart targeting nutraceutical. In some embodiments, the kidney or cardiovascular targeting compound is a low-molecular-weight peptide. In some embodiments, the peptide is a Natriuretic peptide (or analog), that binds to the atrial natriuretic peptide receptor in the kidney or cardiovascular system [see Massimo et al. 2014. European Heart Journal, 35, 419-425].

In some embodiments, the targeting nutraceutical is an inflammation-targeting nutraceutical.

In some embodiments, the inflammation-targeting nutraceutical is Isoflavone (Phytoestrogen), in soy foods.

In some embodiments, the ligand attached to the NP is genistein or daidzein, two isoflavones from soy which affect on rheumatoid arthritis (RA).

One of the possible mechanisms of genistein and daidzein action is their estrogen-like effects. Genistein is structurally related to estrogen and binds estrogen receptors. It has already been demonstrated that estrogen-like sexual hormones improve and prevent RA status (McMurray 2001). It is also possible that the isoflavones exert their effects through a direct effect on cartilage metabolism. Both estrogen a and p receptors can be found in the cartilage of human joints, so cartilage may also be a target for modulators of estrogen receptors. Intra-articular injection of estrogen leads to the destruction of matrix collagen by destroying lactate dehydrogenase enzyme in chondrocytes. Estrogen-like isoflavones that bind to estrogen receptors and interfere with local estrogen function can be effective in osteoarthritis. [See Mohammad-Shahi, Majid et al. “Comparison of the effects of genistein and daidzein with dexamethasone and soy protein on rheumatoid arthritis in rats.” BioImpacts. BI vol. 1, 3 (2011): 161-70.]

In some embodiments, the inflammation-targeting nutraceutical is flavonoids and other phytochemicals. Flavonoids and other phytochemicals from oregano were characterized for their agonist and antagonist PPARγ activities.

The peroxisome proliferator activated receptors (PPARs) are a subset of the nuclear receptor superfamily. Unlike the classical hormone-activated receptors such as the estrogen receptor, which is located in the cytoplasm and translocates to the nucleus after binding of the activating ligand, the PPAR receptors reside in the nucleus bound to DNA response element

Through their binding to PPARs—or directly by their binding to NF-kB and AP-1—dietary molecules can inhibit the inflammatory process.

Dietary phytochemicals with quite different structures and properties from the unsaturated fatty acids discussed so far, have been characterized from oregano by activity-based fractionation. Compounds were first identified as ligands using a PPARγ fluorescence-polarization assay and subsequently classified as agonist or antagonist, based on their ability to inhibit rosiglitazone-induced cellular responses.

In some embodiments, the NP is the dietary flavonoids quercetin, luteolin, rosmarinic acid and diosmetin. The dietary flavonoids were found to be PPARγ antagonists, naringenin and apigenin were modulators and biochanin A was found to be a PPARγ agonist [see Martin, H. (2010). Role of PPAR-gamma in inflammation. Prospects for therapeutic intervention by food components. Mutation Research/Fundamental and Molecular Mechanisms of Mutagenesis, 690(1-2), 57-63.]

Although the bioavailability of these phytochemicals needs to be addressed, the approach demonstrates that many common phytochemicals have the potential to influence PPARγ activity, due to the promiscuous nature of the binding site. Zingerone, the active ingredient in ginger, which is structurally similar to curcumin, is also anti-inflammatory through its ability to increase PPARγ expression.

In some embodiments, the inflammation-targeting nutraceutical is ginsenoside.

Ginsenoside Re ameliorates inflammation by inhibiting the binding of lipopolysaccharide to TLR4 on macrophages [see Lee, In-Ah, et al. “Ginsenoside Re ameliorates inflammation by inhibiting the binding of lipopolysaccharide to TLR4 on macrophages.” Journal of agricultural and food chemistry 60.38 (2012): 9595-9602.] Ginsenoside Rb1 and its metabolite compound K inhibit IRAK-1 activation—The key step of inflammation [see Joh, Eun-Ha, et al. “Ginsenoside Rb1 and its metabolite compound K inhibit IRAK-1 activation—the key step of inflammation.” Biochemical pharmacology 82.3 (2011): 278-286.]

In the preliminary study, ginsenoside Rb1, a main constituent of the root of Panax ginseng (family Araliaceae), and its metabolite compound K inhibited a key factor of inflammation, nuclear transcription factor κB (NF-κB) activation, in lipopolysaccharide (LPS)-stimulated murine peritoneal macrophages. Ginsenoside Rb1 and compound K also significantly inhibited the activation of interleukin-1 receptor-associated kinase-1 (IRAK-1), IKK-β, NF-κB, and MAP kinases (ERK, INK, and p-38).

In some embodiments, the inflammation-targeting nutraceutical is c-phycocyanin Biliprotein from Spirulina platensis C-phycocyanin is a selective inhibitor of cyclooxygenase-2. (COX-2) with a very low IC50 COX-2/IC50 COX-1 ratio (0.04). The extent of inhibition depends on the period of preincubation of phycocyanin with COX-2, but without any effect on the period of preincubation with COX-1. [see Reddy, C. Madhava, et al. “Selective inhibition of cyclooxygenase-2 by C-phycocyanin, a biliprotein from Spirulina platensis.” Biochemical and biophysical research communications 277.3 (2000): 599-603.]

In some embodiments, the targeting nutraceutical is a viral-inflammation targeting nutraceutical.

In some embodiments, the viral-inflammation targeting nutraceuticals are fucoidans.

Antiviral activity is ensured by the binding of fucoidans to the CD4 glycoprotein on T lymphocytes, an essential immunoglobulin in the infection process of host cells by the viruses [see Chollet, Lucas, et al. “Fucoidans in nanomedicine.” Marine drugs 14.8 (2016): 145.]

The mode of action of the galactofucan was shown to be the inhibition of viral binding and entry into the host cell Furthermore, algal fucoidans have been found to suppress IgE and Th2 cytokine production as shown in recent studies.

Therapeutic activity: In some embodiments, the complex comprises at least one Active Pharmaceutically Ingredient (API). An API is a compound that has a pharmacological (or therapeutic) effect. In some embodiments, the API is a synthetic compound, a natural product, a semi-natural product or a neurochemical. In some embodiments, the API is an active nutraceutical. In some embodiments, at least one API is bound/attached to the outer surface/shell of the NP, contained with the interior volume of the nanoparticle or bound within the NP (within the lipid layer of the NP). In some embodiments, the complex comprises a linker that binds to the ligand and to the API.

The term “active nutraceutical ingredient (ANI)” refers to a compound that is derived or originated from a natural source and has been clinically shown to be beneficial in the prevention and/or treatment of a disease, disorder or condition of a subject suffering therefrom (see also definition in Chao et al. (2012) Nutrition Reviews, 70(7), 373-386). The ANI can be targeted to various systems and disorders, such as the central or peripheral nervous system (such as in Parkinson's disease, Alzheimer disease or multiple sclerosis), neo-vascularization or neoplasmic malignant growth (solid cancer tumors), endocrine system (such as diabetics) or the gastrointestinal system (such as gastric ulcers or IBD).

In some embodiments, the active nutraceutical is a neuro-active nutraceutical:

In some embodiments, at least one neuro-active nutraceutical is selected from polyphenols, including simple polyphenols and phenolic acid derivatives, such as Gallic acid, Curcumin, Ferulic acid, Rosmarinic acid, and Verbascoside. The nutraceutical can be selected from complex polyphenols including Flavonoids (such as Catechin, Epicatechin, EGCG, Kaempferol, Morin, Myricetin or Quercetin), Flavones (such as Apigenin, Scutellarein and Baicalein), Isoflavone (such as Genistein), Anthocyanidins (such as Delphinidine and Proanthocyanidins), Tannins (such as Tannic Acid), Stilbenes (such as Piceatannol, Piceid, Resveratrol, Viniferin), omega fatty acids (such as eicosapentaenoic (EPA) and docosahexaenoic (DHA)) and Lignans (such as Nordihydroguaiaretic acid (NDGA)). The (neuro)-active nutraceutical can also be sugar alcohol like mannitol, sorbitol, and any combinations thereof.

In some embodiments, the neuro-active nutraceutical has anti-oxidative properties and neuroprotective properties.

In some embodiments, the active nutraceutical is an onco-active nutraceutical:

In some embodiments, the onco-active nutraceutical can control the growth of cancer cells. In some embodiments, the onco-active nutraceutical is selected from a group consisting of curcumin, oleic acid. In some embodiments the active nutraceutical is a spice extract, selected from ajoene, allicin, allyl isothiocyanate, bergapten, β-carotene, α-sitosterol, canthaxanthin, capsaicin, carnosol, cineole, cinnamaldehyde, citral, crocin, curcumin, diallyl disulfide, diallyl sulfide, diallyl trisulfide, [6]-gingerol, kaempferol, limonene, lutein, myristicin, [6]-paradol, rosmarinic acid, S-allyl cysteine, S-allyl mercapto cysteine, sesamin, sesamolin, shogaol, sulforaphane, terpinene-4-ol, turmerone, and ursolic acid.

In some embodiments, the onco-active nutraceutical is a dye such as an alkannin (or an analog or enantiomer of alkanes). In some embodiments, the onco-active nutraceutical is a plant hormone, such as Jasmonic acid (JA) (shown to induce death in lymphoblastic leukemia cells) or a derivative of, such as Methyl jasmonate (shown to inhibit proliferation in of cancer cell lines).

In some embodiment, the active nutraceutical regulates the BcL-2 protein.

In some embodiments, the targeting nutraceutical is an endo-active nutraceutical:

In some embodiments, the endo-active nutraceutical can regulate the endocrinological system is a polyphenol selected from a group consisting of stilbenes or stilbenoids (such as Piceatannol and Resveratrol). In some embodiments, the active nutraceutical is hormone (such as Insulin).

In some embodiments, the active nutraceutical is a gastro-active nutraceutical, active in the gastrointestinal tract, such as the stomach or intestine.

In some embodiments, the gastro-active nutraceuticals are fat-soluble vitamins (such as A, D, E, and K). In some embodiments, the gastro-active nutraceuticals are lipids, such as polyunsaturated fatty acids (PUFA). The PUFA can be n-6 PUFA arachidonic acid (AA), which is the precursor of inflammatory eicosanoids like prostaglandin E(2) and leukotriene B(4), and the n-3 PUFAs eicosapentaenoic acid (EPA) and docosahexaenoic acid (DHA), which are abundant in fish oils. Lipids and fat-soluble vitamins can be dissolved in the lipid (bi) layer of the NP. In some embodiments, the NP can comprise a combination of Vitamins and PUFAs.

In some embodiments, the gastro-active nutraceutical is a polyphenol such as is Stevia, a food additive extracted from the leaves of Stevia rebaudiana. Its bioactive elements and/or metabolites include steviol, steviol glycosides, mainly rebaudioside A and stevioside. In some embodiments, the polyphenol is a cyclic or linear (curcuminoid) diarylheptanoids, such as curcumin. In some embodiments, the polyphenol is a green tea flavonoid, such as epigallocatechin-3-gallate (EGCG).

Unless otherwise stated, with reference to numerical quantities, the term “about” refers to a tolerance of 25% of the stated nominal value.

Unless otherwise stated, all numerical ranges are inclusive of the stated limits of the range.

Reference is now made to a composition for delivering an active pharmaceutical ingredient (API) to a targeted tissue or a targeted cell 22 of a mammalian body, comprising a complex comprising of: at least one nanoparticle 10; at least one API; the API is contained by the nanoparticle 10; at least one nutraceutical ligand 13, the nutraceutical ligand 13 is configured to specifically bind to the targeted tissue or the targeted cell 22; characterized be the API being activated at the targeted tissue or the targeted cell 22 by the mediation of the nutraceutical ligand 13.

Further reference is made to the above-mentioned composition as defined above, further characterized by the composition further comprising a linker 17 that binds the at least one nutraceutical ligand 13 to the at least one nanoparticle 10.

Further reference is made to the above-mentioned composition as defined in any of the above, further characterized by the nutraceutical being a targeting nutraceutical.

Further reference is made to the above-mentioned composition as defined in any of the above, further characterized by the nanoparticle 10 being selected from the group consisting of a liposome, a micelle, a solid-lipid nanoparticle, a cyclodextrin nanoparticle, a dendrimer, a polymeric nanoparticle, a micro/nano-emulsion, and any combinations thereof.

Further reference is made to the above-mentioned composition as defined in any of the above, further characterized by the micelle being a mono-layer or a bi-layer 11 construction.

Further reference is made to the above-mentioned composition as defined in any of the above, further characterized by the nanoparticle 10 being in the range of 10-250 nm.

Further reference is made to the above-mentioned composition as defined in any of the above, further characterized by the API being bound to the nanoparticle 10 via a chemical or a physical bond.

Further reference is made to the above-mentioned composition as defined in any of the above, further characterized by the API 14 being contained within the mono-layer or bi-layer 11, API 15 being contained between layers of the bi-layer 11, or API 16 being bound to an external layer of the mono-layer or bi-layer 11.

Further reference is made to the above-mentioned composition as defined in any of the above, further characterized by the chemical or physical bond being selected from the group consisting of a covalent bond, a hydrogen bond, a wan der Waals bond, a hydrophobic bond, an electrostatic bond, a London bond, a ionic bond, a salt bridge, adsorption, and any combination thereof.

Further reference is made to the above-mentioned composition as defined in any of the above, further characterized by the composition being adapted to release the API at the targeted tissue or the targeted cell 22.

Further reference is made to the above-mentioned composition as defined in any of the above, further characterized by the API being selected from a group consisting of a nutraceutical, a small molecule, a food supplement, a protein, a hormone, a peptide, and a fatty acid.

Further reference is made to the above-mentioned composition as defined in any of the above, further characterized by the nutraceutical being an active nutraceutical ingredient (ANI).

Further reference is made to the above-mentioned composition as defined in any of the above, further characterized by the ANI being selected from a group consisting of a neuro-active nutraceutical, an onco-active nutraceutical, an endo-active nutraceutical, a gastro-active nutraceutical, and any combination thereof.

Further reference is made to the above-mentioned composition as defined in any of the above, further characterized by the ANI being curcumin.

Further reference is made to the above-mentioned composition as defined in any of the above, further characterized by the API being generally recognized as safe (GRAS).

Further reference is made to the above-mentioned composition as defined in any of the above, further characterized by the nutraceutical ligand 13 being selected from a group consisting of a neuro-targeting compound, a tumor-targeting compound, a gastric targeting compound, an endo (pancreatic)-targeting compound, a cardiovascular targeting compound, a kidney targeting compound, an inflammation targeting compound, and any combination thereof.

Further reference is made to the above-mentioned composition as defined in any of the above, further characterized by the neuro-targeting compound being selected from a group consisting of caffeine, theobromine (xantheose), paraxanthine, theophylline, 1,3,7-trimethyluric acid, guarana (Paullinia cupana, HBK), phenethylamine (PEA, chocolate), tryptamine (Acacia), and any combinations thereof.

Further reference is made to the above-mentioned composition as defined in any of the above, further characterized by the neuro-targeting compound being selected from a group consisting of baicalein, wogonin, oroxylin A, and any combination thereof.

Further reference is made to the above-mentioned composition as defined in any of the above, further characterized by the nutraceutical ligand 13 being generally recognized as safe (GRAS).

Further reference is made to the above-mentioned composition as defined in any of the above, further characterized by the composition being configured to achieve at least one of the following: a higher concentration of the API at said target tissue; a lower concentration of the API in non-targeted tissue; a higher activity of the API at said target tissue; an extended activity of the API at the target tissue; an increased delivery of said API to a target cell 22.

Further reference is made to the above-mentioned composition as defined in any of the above, the delivery mechanism of the API further being characterized as an active or a passive uptake mechanism.

Further reference is made to the above-mentioned composition as defined in any of the above, further characterized by the uptake mechanism of the API being defined as the uptake of the at least one nanoparticle 10 containing the at least one API, or of the at least one API released from the at least one nanoparticle 10.

Further reference is made to the above-mentioned composition as defined in any of the above, further characterized by the uptake mechanism of the at least one nanoparticle 10 containing the at least one API being endocytosis.

Further reference is made to the above-mentioned composition as defined in any of the above, further characterized by the mode of the endocytosis being selected from the group consisting of receptor-mediated endocytosis, clathrin-mediated endocytosis, non-mediated endocytosis, caveolae, pinocytosis and phagocytosis.

Further reference is made to the above-mentioned composition as defined in any of the above, the active uptake mechanism further characterized as being facilitated by one of the group consisting of transporters, pumps and channels.

Further reference is made to the above-mentioned composition as defined in any of the above, further characterized by the passive uptake mechanism being selected from the group consisting of diffusion, facilitated diffusion and filtration.

Further reference is made to the above-mentioned composition as defined in any of the above, further characterized by the uptake being selected from the group consisting of secondary active transport, coupled transport or co-transport.

### EXAMPLES

### Example 1: Preparation of the Nanoparticle Complex

Liposomes containing targeting nutraceutical covering a core containing an active nutraceutical are manufactured by formulating a self-assembling amphiphilic biocompatible lipid, such as glyceryl monooleate (GMO), loaded with an active nutraceutical and “decorated” with targeting nutraceutical. The liposome can be produced by sonication, extrusion, reverse-phase evaporation, and solvent injection. Production may further include separation of the produced liposomes by size.

Cellular Uptake Study [as described in Ismail et al., 2013. International Journal of Nanomedicine, 8, 393-406]. Cellular uptake of the drug-loaded NPs is essential for attaining ample drug level to elicit a substantial therapeutic response.

ADME (Targeting: Absorption, Distribution and accumulation studies—NPs (liposomes) loaded with a targeted nutraceutical, are marked with a fluorescence dye, or radioactive (3H, 14C) liposomes. The loaded liposomes are injected into the mice (Intravenously, via the gastrointestinal cavity, subcutaneously) and scanned to identify and measure the accumulation of the fluorescence dye in target tissues.

### Example 2: Neurological Function

Cell Viability Study are conducted to assess the effect of decorated liposomes on rotenone-induced toxicity, via a cell viability study by 3-(4,5-dimethylthiazol-2-yl)-2,5-diphenyltetrazolium bromide (MTT) assay was performed in PC12 cells. The cytotoxicity study with rotenone should reveal a dose dependency with 2 μg/mL of rotenone causing approximately 50% cell death. Further, to test the protective effect of decorated liposomes on rotenone-induced toxicity, we will expose the PC12 cells to rotenone (2 μg/mL) along with various concentrations of caffeine/baicalein/wogonin/oroxylin A mannitol, or both, native or in liposomal nanoformulations.

As depicted in FIG. 3, caffein-decorated SNLs containing curcumin were shown not to be toxic.

Behavioral Experiments [as described at Ismail et al. 2013. International Journal of Nanomedicine, 8 393-406]

Morris water maze—After 3 months, spatial memory is be measured by the Morris water-maze (MWM) test. The water maze consisted of a circular water tank (160 cm in diameter and 35 cm in height), which is divided by four fixed points on its perimeter to four quadrants. It contains an escape platform of 10 cm in diameter of the same color as the rest of the basin (to eliminate any false-positive results due to vision), placed in a constant quadrant of the basin throughout the trials and kept 1.5 cm below the water surface. Rats are placed at a start point in the middle of the rim of a quadrant not containing the escape area with their face to the wall. Animals have four trials per day separated by 10 minutes for 5 successive days, during which the times required to find the hidden platform which are be averaged.

Additional studies include, lipid-based liposomes, restoration of functional deficits in a Rotenone-induced mouse model of PD, measurement of intracellular oxidative stress, induction of Autophagy-Lysosome functions (The autophagy-lysosome pathway (ALP) is a vital mechanism for the removal of abnormal and aggregated proteins, and ample of evidence suggests an impairment in this pathway, which thereby aggravates disease progression), modulation of apoptosis, plasma bioavailability and brain bio-distribution of mannitol loaded and caffeine decorated liposomes, restoration of functional deficits in a Rotenone-induced mouse model of PD among other possible research experiments.

Parkinson's Disease: Evaluation of PD treatments is be evaluated in both 6-OHDA and MPTP Parkinson's disease models, administering the NP to mice (n=8). The mice receive four intraperitoneal (i.p.) injections of MPTP-HCl (18 mg/kg of free base) in saline at 2-h intervals. The intoxicated mice are treated daily with 100 μL NP-based composition comprising a neuro-targeting nutraceutical and a neuro-active nutraceutical from 3 h after the last injection of MPTP via gavage using a gavage needle.

Two types of behavioral experiments are conducted: open field experiment for locomotor activity and a rotarod experiment for feet movement. Locomotor activity is measured 7 days after the last dose of MPTP injection in a Digiscan Monitor (Omnitech Electronics, Inc., Columbus, OH). This Digiscan Monitor records stereotypy and rearing, behaviors that are directly controlled by the striatum, as well as other basic locomotion parameters, such as horizontal activity, total distance traveled, number of movements, movement time, rest time, mean distance, mean time, and center time. Before any insult or treatment, mice are placed inside the Digiscan Infra-red Activity Monitor for 10 min daily and on a rotarod for 10 min daily for 3 consecutive days to train them and record their baseline values. Briefly, animals are removed directly from their cages and gently placed nose-first into a specified corner of the open-field apparatus and after release, data acquisition began at every 5 min interval. DIGISCAN software is used to analyze and store horizontal and vertical activity data, which are monitored automatically by infrared beams. In rotarod, the feet movement of the mice is observed at different speeds. To eliminate stress and fatigues, mice are given a 5-min rest interval. Then 7 d after the last dose of MPTP, open field assays and a rotarod tests are carried out twice at 6 h intervals on each mouse separately. Locomotor activity measures are assessed after baseline value comparison.

Multiple sclerosis: 6-week old male DA rats (n=8, weight=200 gr±20 gr) are kept at 23±2 °C with a relative humidity of 50% and lights on at 6 a.m. on and off at 6 p.m. Animals are fed ad libitum with a commercial diet with free access to water. EAE disease is induced by immunization of rats with the encephalitogenic emulsion prepared by mixing equal amounts of freshly extracted DA rat spinal cord homogenate (50% w/v in saline) and complete Freund's adjuvant (CFA) containing 7 mg/ml Mycobacterium tuberculosis. Immunized rats are monitored and neurological scores assessed in an unblinded fashion daily beginning on day 5 post disease induction for clinical symptoms of EAE. Clinical disease is scored for typical signs according to the following scale: 0, no clinical signs; 1, flaccid tail; 2, hind limb weakness; 3, hind limb paresis; 4, complete bilateral hind limb paralysis; 5, death due to EAE]. The NP-based composition comprising a neuro-targeting nutraceutical and a neuro-active nutraceutical is suspended in carboxymethylcellulose made up to 0.5% in water to which Tween 80 is added to reach a final concentration of 0.6% and is administered orally daily for 34 days. For therapeutic treatment, monitoring animals for signs of clinical disease starts five days following disease induction.

Alzheimer's diseases: Experiments are carried out in a double transgenic animal model of AD (overexpressing human APP with the London mutation (V717I) and human PS1 bearing the A246E mutation). 6-month old mice (n=8, weight=animals are treated by intra-cerebroventricular (i.c.v.) infusion and intraperitoneal (i.p.) injection of the NP-based composition comprising a neuro-targeting nutraceutical and a neuro-active nutraceutical for 8 wk. The immunological reaction to the treatment is studied in the i.p. treated animals. Histological analysis is conducted for amyloid plaques, amyloid load in the cerebral cortex and hippocampus, respectively.

### Example 3: Oncological Function

Cancer: In vitro screening of various cancer types, and then test the decorated liposomes in IN VIVO mouse models—xenograft mouse mode for brain glioma, colon cancer, breast cancer. tumor cells (colon cancer) are grown subcutaneously in immune-deficient hosts (severe combined immune deficient (SCID) mice) for 14 days. The tumor is measured and the NP-based composition comprising an onco-target-nutraceutical and an onco-active nutraceutical is injected. The tumor is evaluated for 14 days following treatment.

Glycolysis blocker: to test the effect of the NP on tumor growth in vivo, 6-week-old male SCID mice are injected with B16 melanoma cells (or other cancer cells) subcutaneously (1×106 cells per mouse, 6 mice per group). After the xenografts were established, the tumor-bearing mice are administered with an NP-based composition comprising an onco-target-nutraceutical and an onco-active nutraceutical is injected via intraperitoneal injection. The mice are evaluated for 14 days.

### Example 4: Endocrine Function

Diabetes: chemical models have been shown to be effective for the evaluation of glucose-lowering treatments. STZ [2-deoxy-2-(3-(methyl-3-nitrosoureido)-D-glucopyranose] is administered (via i.p. or i.v.) and enters the pancreatic beta-cell through the Glut-2 transporter and causes alkylation of the DNA (Szkudelski, 2001). Subsequent activation of PARP leads to NAD+ depletion, a reduction in cellular ATP and subsequent inhibition of insulin production (Sandler and Swenne, 1983). A single dose of STZ (150 mg/kg) is administered to mice (n=8). 24 hours following STZ administration, the NP comprising a pancreatic-targeting nutraceutical and a pancreatic-active nutraceutical is administered via i.p. to half of the mice and the blood-glucose levels of both groups are tracked for 10 days.

Wistar-Bratislava albino male rats (n=8, weight 225 gr±25 gr) are kept in polypropylene cages at constant temperature (24±2 °C), 60±5% humidity, and light-dark regime. The rats receive unrestricted access to food and water. The NP comprising a pancreatic-targeting nutraceutical and a pancreatic-active nutraceutical is administered via i.p. 30 min before STZ administration, according to Porfire et al. After 72 h, fasting plasma glucose is checked, and the animals with plasma glucose values of more than 200 mg/dL are considered diabetic (and included in the study).

Pancreatic lipase activity assay: Commercial pancreatic lipase is prepared according to McDougall et al. Pancreatic lipase is prepared and the purity of the lipase and co-lipase is estimated by SDS-PAGE analysis. Protein concentration is determined by Bradford. Pancreatic lipase substrate (pNPL, 0.08% mass per volume) is dissolved in 5 mM sodium acetate solution (pH=5.0, 1% Triton X-100). An NP-based composition comprising a pancreatic-targeting nutraceutical and a pancreatic-active nutraceutical dissolved at three different concentrations in methanol. An Orlistat solution is prepared at 1.2 mg/mL in methanol. Stability of the compounds in the solvent is tested for the time of assay by measuring their UV-Vis spectra.

### Example 5: Gastrointestinal Function

Inflammatory Bowel Disorder (IBD) (TNBS model): 6-8-year-old Wister rats (240-280 g) (n=10) are dosed with 100 mg/kg following a period (24 h) of fasting, to give an average Wallace score of 5. The mice are evaluated 3 and 7 days after dosing. 7 days following TNBS dosing, the mice are treated with an NP-based composition comprising a gastro-targeting nutraceutical and a gastro-active nutraceutical and evaluated for 14 days.

Fatty liver: Non-alcoholic fatty liver disease (NAFLD) is emerging as a common medical problem. Mice are feed a diet that consists of considerable amounts of sucrose (40% of energy), only moderately enriched with fat (10%) and deficient in methionine and choline. Male wild-type (WT) mice C57Bl/6 are fed the MCD diet (MP Biomedicals, #960439; n=15 mice) for 8 weeks. The diet started is started at 12 weeks of age, with the mice treated with an NP-based composition comprising a target-nutraceutical and an active nutraceutical after 8 weeks on the diet (20 week age). Inflammation is tracked for 3 weeks.

Gastric ulcers: Balbc mice (n=8) are infected with H. pylori (Sydney strain) with 0.2 cc of inoculum intragastrically using a 24 gauge×1 in. straight stainless steel feeding tube attached to a 1 cc syringe (approximately 2×108 organisms). The mice receive a total of 3 doses, 1 per day and each separated by 1-2 days. 21 days following infection, the mice are treated with an NP-based composition comprising a gastro-targeting nutraceutical and a gastro-active nutraceutical. The mice are evaluated for 15 days.

Obesity: Zucker Diabetic Fatty (ZDF) rats (n=8, average weight 135 gr±5 gr) were maintained under pathogen-free conditions at 23±0.5 °C and 55%10% relative humidity with a 12 h light-dark cycle and were provided with water and a commercial diet and are evaluated by treating the mice with an NP-based composition comprising a gastro-targeting nutraceutical and a gastro-active nutraceutical for 14 weeks and tracking the weight.

Enzymatic assay of lipase from Human Pancreas: Obesity is a disarray of energy balance and primarily considered as a disorder of lipid metabolism. A growing number of enzymes involved in lipid metabolic pathways are being identified and characterized. They represent a rich pool of potential therapeutic targets for obesity. Inhibition of PL (triacylglycerol acyl hydrolase), the principal lipolytic enzyme, synthesized and secreted by is one of the approaches for the development of newer anti-obesity drugs. Tetrahydrolipstatin (Orlistat), a commercial anti-obesity drug, is a known pancreatic lipase inhibitor. The NP is further characterized by in-vitro lipase activity to access their anti-obesity potential. Measurement of pancreatic lipase activity in-vitro Lipase activity is determined by measuring the rate of release of oleic acid from triolein.

### Example 6: Kidney Function

Kidney functionality: 8-week-old female Wistar rats (n=12, weighing 180 gr±20 gr) are housed in single metabolic cages at constant room temperature (20 °C) and humidity (75%) under a controlled light/dark cycle. The rats are fed a standard chow diet and animals had free access to drinking water. The rats are treated with an NP-based composition comprising a nephrotic-targeting nutraceutical and a nephrotic-active nutraceutical at day 0. 4 rats are sacrificed every 7 days for the morphological studies of the kidneys.

Nephritis: the study is conducted in normotensive female Wistar rats (n=8, weights of 210 gr±20 gr). Immune complex nephritis is induced with the rats receiving an initial subcutaneous injection of 5 mg of incomplete Freund's adjuvant (CFA). At week 3, a second dose is given in incomplete Freund's adjuvant. At week 4, daily intraperitoneal administration of 10 mg ovalbumin is started. Proteinuria should appear, starting at the 9th week. When proteinuria reaches 20 to 50 mg/day, animals are treated orally with NP-based composition comprising a nephrotic-targeting nutraceutical and a nephrotic-active nutraceutical. Three weeks after the onset of proteinuria, animals are sacrificed, blood collected and kidneys removed.

### Example 7: SLN Pre-Concentrate Component

The following materials (Table 1) were used in the preparation of the curcumin-loaded SLN pre-concentrate.

TABLE 1
Materials used in the preparation of the
curcumin-loaded SLN pre-concentrate

| | | Content |
|---|---|---|
| Component | Function | (% w/w) |
| Cremophor RH 40 (PEG-40 | Non-ionic solubilizer/ | 18 |
| Hydrogenated Castor Oil) | emulsifier | |
| Tween 20 (Polysorbate 20) | Non-ionic surfactant/ | 18 |
| | emulsion stabilizer | |
| Span 80 (Sorbitan | Non-ionic co-surfactant/ | 18 |
| Monooleate) | emulsion stabilizer | |
| Solutol HS 15 (Polyoxyl 15 | Non-ionic solubilizer/ | 6 |
| Hydroxystearate) | emulsifier | |
| Propylene Glycol (Propane- | Water miscible solvent | 22 |
| 1,2-diol) | | |
| Cocoa Butter (Theobroma | Solid lipid matrix core | 14 |
| oil) | | |
| Curcumin (from Curcuma | Active pharmaceutical | 4 |
| longa L.) | ingredient | |

### Preparation of the Caffeine-Coated Curcumin-Loaded Sln (“Caffeine-SLN-Curcumin”)

### Step 1: Preparation of the SLN Pre-Concentrate (PNL Pre-Concentrate)

Weighed quantities of Cremophor RH 40, Tween 20, Span 80, Solutol HS 15, propylene glycol, cocoa butter, and curcumin were combined in a clean glass vial according to the weight ratios specified in Table 1. The curcumin content was maintained at 4% w/w; loadings above 4% w/w resulted in curcumin precipitation and phase instability.

The sealed vial was placed in an oil bath or temperature-controlled water bath set to 37-50 °C. The mixture was stirred continuously using a magnetic stir bar at moderate speed for a period of 1-3 hours until complete homogeneity was achieved, as evidenced by a visually uniform, dark-orange oily liquid free of particulate matter.

The resulting pre-concentrate (hereinafter “SLN pre-concentrate” or “PNL pre-concentrate”) is a thermodynamically stable, single-phase mixture that disperses spontaneously in aqueous media upon contact. The pre-concentrate is stable when stored at room temperature (20-25 °C) in the dark, protected from moisture.

### Step 2: Formation of SLN Nanoparticles by Aqueous Dispersion

Prior to dispersion, the SLN pre-concentrate was warmed to 37 °C for a minimum of 15 minutes and mixed by shaking or inversion to ensure complete homogeneity.

Uncoated SLN were prepared by adding 100 μL of the pre-concentrate to 1.0 mL of DDW (or MilliQ water) pre-heated to 37 °C, in a 1:10 v/v ratio of pre-concentrate to aqueous phase (10% v/v pre-concentrate). Immediately upon addition, the mixture was vortexed vigorously for 15-120 seconds until a homogeneous, optically clear to slightly opalescent dispersion was obtained. This step is referred to as the “dispersion step.”

Spontaneous nanoparticle formation occurs as the amphiphilic surfactant-rich pre-concentrate contacts the aqueous medium, driving self-assembly of lipid nanoparticles with the curcumin molecules sequestered within the hydrophobic cocoa butter matrix. No high-pressure homogenization, sonication, or other high-energy input is required; the process is driven entirely by the thermodynamic affinity of the formulation components for ordered interfacial assembly.

### Step 3: Caffeine Surface Covering

Caffeine-decorated SLN (caffeine-SLN) were prepared by replacing the DDW dispersion medium in Step 2 with an aqueous caffeine solution, prepared as follows:

Caffeine was dissolved in DDW or MilliQ water at a concentration of 1.0 mg/mL (0.10% w/v) with gentle warming to 37 °C to assist dissolution.

The caffeine solution was pre-warmed to 37 °C immediately before use

100 μL of the SLN pre-concentrate (warmed and homogenized as described in Step 2) was added to 1.0 mL of the 1.0 mg/mL caffeine solution.

The mixture was vortexed immediately for 15-120 seconds.

Caffeine adsorption onto the SLN surface occurs via non-covalent interactions between the caffeine molecule and the hydrophobic lipid-surfactant interface of the negatively charged SLN. The anionic character of the freshly formed SLN surface (zeta potential −24.3±0.55 mV) facilitates electrostatic and hydrophobic association with caffeine. No covalent bond formation is required and no coupling reagents or reactive chemistries are involved.

The effective caffeine concentration at the particle surface corresponds to approximately 5% w/w relative to the total lipid phase (curcumin plus cocoa butter combined). This ratio is achievable by selecting a caffeine solution concentration of 1.0 mg/mL and maintaining the 1:10 v/v pre-concentrate:aqueous medium ratio as described.

Alternative coating protocol: In a scaled preparation method, 0.010 g of caffeine powder was dissolved directly in 10 mL DDW. One gram of the SLN pre-concentrate was then added and the mixture stirred at 37 °C for 15 minutes. The dispersion was then subjected to centrifugation at 4 °C for 15 minutes. The supernatant (containing unbound caffeine and water-soluble components) was carefully removed, and the pelleted nanoparticle fraction was resuspended in 10 mL of fresh DDW. This alternative protocol produces caffeine-SLN with comparable physicochemical properties and is particularly suited for scaled batches requiring removal of excess unbound caffeine prior to analysis or in vivo administration.

### Step 4: Filtration/Extrusion

For in vivo administration, the freshly prepared SLN dispersion (both uncoated and caffeine-coated) was subjected to filtration through a 0.2 μm hydrophilic PTFE (wwPTFE) syringe spin-filter at 40 °C. This step was performed within 15 minutes of nanoparticle formation.

Filtration at 40 °C (rather than at room temperature) is critical: performing filtration at room temperature leads to lipid solidification at the filter membrane with concomitant curcumin retention. At 40 °C, the lipid matrix remains sufficiently fluid to pass through the membrane.

The filtration step substantially improves the polydispersity index (PDI) of the dispersion, reducing it from approximately 0.31 in unfiltered samples to approximately 0.23 in filtered samples, without measurable loss of curcumin content. Curcumin retention was confirmed by UV-Vis absorbance at the curcumin λmax of 426 nm, measured after complete dissolution of the nanoparticles in absolute ethanol; filtered and unfiltered samples produced equivalent absorbance values at this wavelength.

### Example 8: Physicochemical Characterization of the Caffeine-Coated Curcumin-Loaded SLN

### Particle Size and Polydispersity

The hydrodynamic particle size and polydispersity index (PDI) of the SLN dispersions were measured by dynamic light scattering (DLS) using a Malvern Zetasizer instrument (or equivalent). Measurements were performed at 25 °C or 40 °C as specified, using reduced-volume disposable cuvettes. Each measurement was performed in triplicate and results reported as mean±standard deviation.

For DLS measurement, 100 μL of the SLN dispersion (filtered or unfiltered) was diluted into 1.0 mL of DDW pre-warmed to 37 °C. Alternatively, where precise temperature control was required, measurements were performed directly in the Zetasizer at the target temperature using reduced-volume cuvettes.

Representative physicochemical characterization data for key batches are provided in Table 2:

TABLE 2
Representative physicochemical characterization
data of the coated and uncoated SLN nanoparticles

| | Mean Particle | | Zeta |
|---|---|---|---|
| Batch/Sample Code | Size (nm) | PDI | Potential (mV) |
| Uncoated SLN - 2 h | 149 ± 2.30 | 0.229 ± 0.027 | −24.3 ± 0.551 |
| dispersion (MAE-3- | | | |
| 67-A) | | | |
| Uncoated SLN - | 165 ± 0.35 | 0.438 ± 0.072 | −23.8 ± 0.611 |
| 48 h dispersion | | | |
| (MAE-3-67-A) | | | |
| Uncoated SLN - | ~145-191 | ~0.31 | N/A |
| pre-filtration | | | |
| (LIOS protocol) | | | |
| Uncoated SLN - | ~130-145 | ~0.23 | N/A |
| post-filtration at | | | |
| 40 °C (LIOS) | | | |
| Caffeine-coated | 130 ± 1.09 | 0.418 ± 0.053 | N/A |
| SLN - plasma, 24 h | | | |
| (stability) | | | |
| Caffeine-coated | 245 ± 2.54 | 0.476 ± 0.005 | N/A |
| SLN - acidic pH, | | | |
| 24 h (stability) | | | |

A mean particle size in the range of 130-200 nm with PDI≤0.35 is considered acceptable for both uncoated and caffeine-SLN. Post-filtration PDI≤0.25 is preferred for in vivo preparations.

### Zeta Potential

Zeta potential measurements were performed by electrophoretic light scattering using the Malvern Zetasizer (or equivalent). The SLN dispersions were analyzed in DDW at 25 °C using folded capillary zeta cells. The uncoated SLN exhibited a zeta potential of −24.3±0.55 mV, indicative of a moderately negative surface charge that contributes to colloidal stability through electrostatic repulsion. Zeta potential values more negative than −20 mV are considered adequate for colloidal stability in the present system.

The negative zeta potential of the uncoated SLN surface is a mechanistic prerequisite for caffeine adsorption: caffeine interacts with the anionic lipid-surfactant interface through a combination of hydrophobic and electrostatic forces. A shift in zeta potential toward less negative or slightly positive values upon caffeine decoration may be observed depending on the caffeine concentration and coating protocol used, consistent with partial charge neutralization at the particle surface.

### Morphology

Transmission electron microscopy (TEM) of the uncoated SLN dispersion confirmed the formation of discrete, predominantly spherical nanoparticles consistent with DLS size measurements. No elongated or aggregated structures were observed under these conditions.

### Caffeine Binding Efficiency

The fraction of added caffeine adsorbed onto the SLN surface (caffeine binding efficiency) was quantified by a fluorescence-based assay using 3,5-diaminobenzoic acid (3,5-DABA) as the derivatization reagent.

### Assay Procedure:

1. Caffeine-SLN dispersions were prepared as described above and centrifuged at 8,000 rpm at 4 °C for 15 minutes to pellet the nanoparticles and separate particle-bound from free (unbound) caffeine.
2. The supernatant (containing free caffeine) was collected and diluted as required to fall within the assay linear range.
3. A 3,5-DABA solution (1 mL of 10 μg/mL in water at pH 7) was added to 4 mL of the diluted supernatant sample.
4. After 10 minutes of incubation at room temperature, fluorescence emission spectra were recorded at an excitation wavelength of 280 nm; emission intensity was measured at 414 nm.
5. Caffeine concentration in the supernatant was determined by reference to a calibration curve constructed in the range of 0.5-15 μg/mL caffeine, which showed a linear response (R²>0.99) across this range.
6. Caffeine binding efficiency (%)=100×(caffeine added−free caffeine in supernatant)/caffeine added.

Results are summarized in Table 3:

TABLE 3
Caffeine binding efficiency

| | Theoretical | Free | Binding |
|---|---|---|---|
| | Caffeine | Caffeine | Efficiency |
| Sample | (mg/mL) | (mg/mL) | (%) |
| SLN-Caffeine | 0.500 | 0.180 | ~56 |
| replicate 1 | | | |
| SLN-Caffeine | 0.500 | 0.160 | ~61 |
| replicate 2 | | | |
| SLN-PBS control (no | 0 | 0.038 | N/A |
| caffeine) | | | |
| PBS-Caffeine (no | 0.500 | 0.406 | N/A |
| SLN) | | | |

Caffeine binding efficiency was consistently in the range of 55-61%, indicating that approximately 55-61% of the added caffeine associates with the SLN surface. The SLN-PBS control (particles dispersed in PBS without caffeine) showed negligible fluorescence signal at 414 nm, confirming that neither the particles nor any formulation component interfere with the assay.

The PBS-caffeine control (caffeine in PBS without particles) showed a lower emission intensity than pure PBS, confirming that caffeine quenches 3,5-DABA fluorescence in the linear assay range.

### Caffeine Coating Stability

The stability of the caffeine decoration on the SLN surface was assessed under two physiologically relevant conditions: (i) simulated gastric acid (0.1 M HCl, pH≈2), and (ii) pooled human plasma. Caffeine-SLN were prepared as described in Section 5.3, and then mixed 1:1.5 v/v with either 0.1 M HCl or human plasma. Samples were incubated at 37 °C with orbital shaking at 75 rpm. At specified time points (0, 0.5, 3, 5, and 10 hours), 100 μL aliquots were withdrawn, diluted in 900 μL PBS, and centrifuged at 8,000 rpm at 4 °C for 15 minutes. Free caffeine in the supernatant was quantified by the 3,5-DABA fluorescence assay as described above. All samples were prepared in triplicate. Results are presented in Table 4:

TABLE 4
Caffeine Coating Stability

| Time | Free Caffeine in Plasma | Free Caffeine at pH 2 |
|---|---|---|
| (hours) | (%) | (%) |
| 0 | 45 ± 0 | 45 ± 0 |
| 0.5 | 45 ± 1 | 60 ± 9 |
| 3 | 47 ± 3 | 63 ± 10 |
| 5 | 48 ± 2 | 66 ± 12 |
| 10 | 72 ± 4 | 81 ± 9 |

The caffeine coating demonstrated excellent stability in human plasma: the fraction of free (unbound) caffeine remained essentially unchanged at approximately 45-48% for the first 5 hours of incubation, rising modestly to approximately 72% at 10 hours. This pattern indicates that approximately 52-55% of the caffeine remains particle-associated in plasma for up to 5 hours post-preparation, providing a sufficient window for systemic circulation and tissue biodistribution following intranasal administration.

In acidic medium (pH 2), progressive caffeine release was observed: free caffeine increased from 45% at t=0 to 81% at t=10 hours. This accelerated release at low pH is consistent with acid-induced disruption of non-covalent caffeine-lipid interactions. Correspondingly, particle size in acidic medium increased from 145 nm to 245 nm over 24 hours, indicative of partial particle swelling or aggregation; particle size in plasma remained stable at approximately 130 nm over the same period.

These stability data collectively confirm that the caffeine-SLN retains structural integrity and meaningful surface caffeine decoration under plasma conditions relevant to the intranasal-to-brain biodistribution pathway.

### Example 9: In Vitro Results with Cell Lines

Caffeine-SLN-Curcumin and uncoated SLN-Curcumin (SLN control) formulations were prepared according to the method described in Examples 7 and 8. Prior to cell culture use, all SLN dispersions were passed through a 0.2 μm sterile membrane filter to ensure sterility. Working dispersions were prepared at the time of each experiment by diluting the freshly prepared nanoparticle dispersions in the appropriate cell culture medium to the desired final concentrations.

All treatment concentrations are expressed as micrograms of curcumin per milliliter (μg/mL), calculated based on the nominal curcumin loading of 4% w/w in the pre-concentrate. Cell Lines and Culture Conditions. Human astrocytoma cells (U-87 MG cell line) and human neuroblastoma cells (SH-SY5Y cell line) were used as the primary cell models. U-87 MG cells were maintained in RPMI 1640 medium; SH-SY5Y cells were maintained in DMEM medium. Both media were supplemented with 10% fetal bovine serum (FBS), streptomycin (100 mg/mL), penicillin (100 U/mL), and Nystatin (12.5 U/mL). Cells were incubated at 37 °C in a humidified atmosphere containing 5% CO₂ and maintained in 75 cm² cell culture-treated flasks. All media and supplements were obtained from Biological Industries, Israel. In addition to the SLN formulations, a caffeine-only control and a double-distilled water (DDW) vehicle control were included in all experiments.

MTT Viability Assay. Cell viability following SLN treatment was assessed using a commercially available MTT assay kit (Abcam, catalogue no. ab146345), performed according to the manufacturer's instructions. Cells were seeded in 96-well plates at a density of 2×10⁴ cells/well (n=4 per group) and allowed to adhere overnight. Following overnight stabilization, cells were exposed to SLN-Curcumin (SLN control) or Caffeine-SLN-Curcumin at curcumin-equivalent concentrations of 5, 10, and 20 μg/mL for U-87 MG cells, and 3.125, 6.25, 12.5, 25, and 50 μg/mL for SH-SY5Y cells. After 24 hours of treatment, the medium was removed and replaced with serum-free medium containing 0.5 mg/mL MTT reagent. Cells were incubated with MTT reagent for 4 hours at 37 °C. The resulting formazan crystals were dissolved in isopropanol containing 0.04 mol/L HCl, and optical density was measured at 570 nm using a Spark spectrophotometer reader (Tecan). Results were expressed as percentage viability relative to untreated control cells.

Intracellular Fluorescence Analysis. At designated time points, cells growing in 96-well plates were washed once with PBS×1 to remove extracellular and surface-bound curcumin, and lysed with RIPA buffer to release intracellular curcumin. Intracellular curcumin content was quantified fluorometrically at excitation/emission wavelengths of 485/530 nm, exploiting the intrinsic fluorescence of curcumin. Results are expressed in arbitrary fluorescence units (A.U.), mean±SEM, n=4 per group.

Adenosine A2A Receptor Blocking Experiment. To confirm that the caffeine-dependent enhancement of intracellular curcumin delivery is mediated through adenosine A2A receptor interaction, a receptor pre-saturation experiment was performed. U-87 MG cells were pre-incubated with free caffeine at a concentration of 10 μg/mL for one hour prior to treatment with SLN-Curcumin or Caffeine-SLN-Curcumin. Intracellular curcumin content was then quantified as described above. Pre-treatment with free caffeine was expected to saturate A2A receptors, thereby reducing receptor-mediated uptake of the caffeine-decorated SLN.

Confocal Imaging Analysis. For fluorescence imaging, cells were seeded in 24-well plates on 12 mm round glass cover slips at a density of 8×10⁴ cells/well and allowed to adhere overnight. Following treatment, cells were washed once with PBS×1 and fixed with 3% paraformaldehyde for 20 minutes at room temperature. Cell nuclei were stained with Hoechst 33342. Imaging was performed on a Nikon A1R confocal microscope at 60× magnification. Curcumin was detected in the GFP channel (green fluorescence); nuclei in the DAPI channel (blue fluorescence).

As depicted in FIGS. 4a-d, after 1 h (FIG. 4b) and 1.5 h (FIG. 4c-d) of treatment of Human Astrocytes (U-87 MG cell line), a significant increase in the curcumin fluorescence was observed following SLN+Caffeine (Caffeine-SLN-Curcumin) sample, as compared to the ligand-less SLN control sample and to SLN-less caffeine sample (Uncoated SLN-curcumin). This indicates that the coating of the sample with caffeine augments the intracellular content of free curcumin 14 at 1.5 hours. In addition, as shown in FIG. 4e, blocking of A2R receptors with an antagonist to caffeine, reduces the curcumin content to the same levels as the SLN control (in the concentration of 20 mg/ml). In fluorescent imaging of said cell line, significantly higher curcumin 32 staining can be observed in the SLN+Caffeine (Caffeine-SLN-Curcumin) treatment at 1.5 hours (FIG. 6a), compared to SLN 10 control (FIG. 6b) and untreated cells 30 (FIG. 6c). In untreated cells 30, green color is not detectable. In SLN 10 control cells 30, certain amount of green color is detectable around the nuclei 31 of the cells 30. In caffeine-decorated cells 30, the green color is so strong it even interferes with the blue color of the nucleus 31.

In another cell line comprising dopamine receptors, Human Neuroblastoma cells (SH Sy5Y), as depicted in FIG. 5a, after 1 hour, a significant increase in the curcumin 14 fluorescence was observed following SLN+Caffeine sample (Caffeine-SLN-Curcumin), as compared to the SLN 10 control sample (Uncoated SLN-curcumin) and to free curcumin 14 sample. However, as depicted in FIG. 5b, after 3.5 hours of treatment, the fluorescence of the SLN 10 control sample Uncoated SLN-curcumin and the free curcumin 14 sample remained at the same levels, as opposed to the SLN+caffeine sample (Caffeine-SLN-Curcumin), which diminished considerably.

### Example 10: In Vivo Pharmacokinetic Study in Rats—Intranasal Administration

### Study Design and Objectives

A controlled, comparative in vivo pharmacokinetic study was conducted to assess the brain biodistribution of curcumin following intranasal administration of three formulations: (i) free curcumin suspension (control), (ii) uncoated SLN-curcumin, and (iii) caffeine-SLN-curcumin. The primary objective was to determine whether caffeine surface decoration conferred differential accumulation of curcumin in the striatum (basal ganglia) relative to the uncoated SLN and free curcumin control. All animal procedures were approved by the Ethics Committee of the Latvian Food and Veterinary Service and were performed exclusively by licensed personnel.

### Formulation Preparation for In Vivo Use

SLN formulations (uncoated and caffeine-decorated) were prepared using the protocol described above. Pre-concentrate vials were warmed to 40 °C for 2 hours with periodic agitation to ensure complete homogenization prior to dispersion. Nanoparticles were prepared 15 minutes before the start of administration and filtered through 0.2 μm wwPTFE spin-filters at 40 °C. Each Eppendorf tube containing the final formulation was briefly vortexed immediately before each intranasal administration to ensure formulation homogeneity.

Free curcumin control suspension was prepared by dissolving curcumin in PEG 400 and adding this solution to a 2.5 mg/mL aqueous carboxymethyl cellulose (CMC) solution to yield a final curcumin concentration of 4.00 mg/mL.

All three formulations were prepared to deliver equivalent curcumin dose per administration volume.

### Animal Model and Administration

Male Wistar rats aged 9-13 weeks, with body weight of 424±29 grams, were used. Animals were randomly allocated to 12 experimental groups: four time-point groups for each of the three formulations (n=4 per group; total N=48 animals). The three formulations were:
Group A: Free curcumin suspension (negative control)
Group B: Uncoated SLN-curcumin
Group C: Caffeine-SLN-curcumin

Formulations were administered intranasally at a volume of 40 μL per nostril (80 μL total) using a calibrated pipette. This corresponds to a curcumin dose of approximately 0.75 mg/kg body weight. At designated time points following administration, animals were euthanized and biological samples collected. The SLN particles as described above has been diluted 1:10 in water immediately before use and administered as intranasal drops.

### Tissue Collection

Tissue collection was performed at four time points 5, 15, 30, and 60 minutes post administration-. At each designated time point, animals were euthanized and the striatum, frontal cortex tissues and plasma were collected.
Plasma: 50 μL of blood was collected by cardiac puncture, centrifuged to obtain plasma, and immediately frozen at −80 °C until analysis.
Striatum (basal ganglia): Dissected under standardized anatomical landmarks, immediately weighed, transferred to homogenization vials, and frozen at −80 °C.
Frontal cortex: Dissected, immediately weighed, transferred to homogenization vials, and frozen at −80 °C.

### Curcumin Extraction and Quantification

### Tissue Preparation:

Frozen striatum and frontal cortex samples were defrosted on ice, transferred to bead homogenizer vials, and weighed.

MilliQ water was added to each tissue sample at a tissue:water ratio of 1:4 (w/v). Samples were cooled to 4 °C and homogenized using a bead mill homogenizer.

Curcumin was extracted by mixing 100 μL of tissue homogenate with 550 μL of ethyl acetate. Plasma extraction was performed by mixing 50 μL of plasma with 230 μL of ethyl acetate. Samples were vortexed vigorously to facilitate extraction, then centrifuged to promote phase separation. The organic (ethyl acetate) layer (500 μL for tissue, 230 μL for plasma) was carefully transferred to fresh Eppendorf tubes.

Solvent was evaporated under vacuum using a centrifuge-type vacuum evaporator (Genevac EZ-2 Plus or equivalent) in low boiling-point mode, maximum 30 °C, to protect curcumin from thermal degradation.

Dried residues were reconstituted in 100 μL of HPLC-grade methanol for UPLC-MS/MS analysis.

### UPLC-MS/MS Analysis:

Curcumin concentrations in all matrices were determined by ultra-performance liquid chromatography coupled with tandem mass spectrometry (UPLC-MS/MS) on a Waters Acquity UPLC H-Class system equipped with a Waters Acquity UPLC BEH C18 column (2.1×50 mm, 1.7 μm particle size), operated at 40 °C. Mobile phase A: 0.1% v/v formic acid in water; mobile phase B: acetonitrile. Gradient elution: 0-1.5 min, 5-98% B; 1.5-3.0 min, 98% B; 3.0-3.5 min, 98-5% B; 3.5-5.0 min, 5% B. Mass spectrometric detection: Waters Xevo TQ-S triple quadrupole detector, ESI positive ionization mode, capillary voltage 3.0 kV. Standard curves were prepared by spiking blank plasma or brain tissue homogenate with known quantities of pure curcumin and subjecting to the extraction procedure described above. The analytical method showed linear response in the range of 0.1-20 ng/mL in all three matrices. All samples were analyzed in duplicate.

### Pharmacokinetic Results

### Plasma Pharmacokinetics

Curcumin plasma concentrations were highest at the earliest measured time point (t=5 minutes) for all three nanoparticle-based formulations. At t=5 minutes Uncoated SLN-Curcumin (Group B) produced a mean plasma curcumin concentration of approximately 85 ng/mL and Group C (Caffeine-SLN-Curcumin) produced approximately 45 ng/mL, compared to approximately 3.7 ng/mL for the free curcumin control representing approximately between 10 and 25 fold higher plasma exposure, respectively, relative to unformulated curcumin. Both SLN formulations demonstrated substantially superior systemic bioavailability relative to the free drug control at this early time point. By t=15 and t=30 minutes, plasma concentrations in all groups converged toward baseline levels. A secondary rebound in plasma curcumin was observed at t=60 minutes, most prominently in the Curcumin and caffeine NPs group (~8.5 ng/mL), consistent with a delayed secondary absorption route discussed below.

### Brain Biodistribution—Striatum (Basal Ganglia) and Frontal Cortex

Both SLN formulations (uncoated and caffeine-coated) produced measurable curcumin concentrations in the striatum and frontal cortex at the earliest assessed time points, confirming that the SLN platform enables rapid brain delivery of curcumin via the intranasal route. Administration of free curcumin resulted in no detectable curcumin in either brain region at those same early time points.

TABLE 5A
Curcumin Concentrations in Striatum (Basal Ganglia) - Approximate
Mean ± Range (ng/g tissue, n = 4 per group)

| | Control | (Uncoated | (Caffeine- | Caffeine- |
|---|---|---|---|---|
| Time Post- | (Free | SLN with | SLN- | SLN-Curcumin/ |
| Administration | Curcumin) | Curcumin) | Curcumin) | Uncoated Ratio |
| t = 5 min | ND | ~1.5 ± 0.8 | ~1.5 ± 0.5 | — |
| t = 15 min | ND | ~1.6 ± 1.6 | ~0.4 ± 0.3 | — |
| t = 30 min | ND | ~0.5 ± 0.3 | ~0.1 ± 0.1 | — |
| t = 60 min ♦ | ~0.7 ± 0.3 | ~0.8 ± 0.4 | ~2.4 ± 0.6 | ~3× |

ND = Not Detectable (below LLOQ of 0.1 ng/mL). Values estimated from UPLC-MS/MS analysis.
♦ Key time point of differential accumulation; highlighted cells denote the Caffeine-SLN advantage.

TABLE 5B
Curcumin Concentrations in Frontal Cortex - Approximate
Mean ± Range (ng/g tissue, n = 4 per group)

| | Control | (Uncoated | (Caffeine- | Caffeine-SLN- |
|---|---|---|---|---|
| Time Post- | (Free | SLN with | SLN- | Curcumin/ |
| Administration | Curcumin) | Curcumin) | Curcumin) | Uncoated Ratio |
| t = 5 min | ~0.5 ± 0.3 | ~5.5 ± 3.5 | ~3.8 ± 2.0 | — |
| t = 15 min | ND | ~0.8 ± 0.5 | ~0.2 ± 0.2 | — |
| t = 30 min | ND | ~0.1 ± 0.1 | ~0.1 ± 0.1 | — |
| t = 60 min ♦ | ~0.7 ± 0.3 | ~0.8 ± 0.4 | ~7.0 ± 2.5 | ~9× |

ND = Not Detectable.
♦ At t = 60 min, Caffeine-SLN-Curcumin exceeded Uncoated SLN-Curcumin by approximately 9-fold in the frontal cortex, the largest differential observed across all matrices and time points.

Tables 5A and 5B collectively establish that caffeine surface decoration confers a time-dependent, region-selective enhancement of curcumin deposition in both striatal and frontal cortical tissue at t=60 minutes post-intranasal administration—a differential of approximately 3-fold in the striatum (basal ganglia) and approximately 9-fold in the frontal cortex relative to uncoated SLN—that is not attributable to nanoparticle encapsulation alone.

Importantly, at 60 minutes following intranasal administration, curcumin concentrations in the striatum of animals receiving Caffeine-SLN-Curcumin were significantly higher than those measured in animals receiving either Uncoated SLN with Curcumin or free curcumin suspension. This differential striatal accumulation—with Caffeine-SLN-Curcumin outperforming both the matched uncoated nanoparticle control and the free drug control in the basal ganglia specifically—provides direct in vivo evidence that caffeine surface decoration actively promotes region-selective curcumin deposition in striatal tissue, beyond the general brain-delivery advantage attributable to nanoparticle encapsulation alone.

This finding is mechanistically consistent with the high density of adenosine receptors (A1, A2A) expressed in striatal neurons and glia, for which caffeine is a known competitive antagonist. The inventors propose that caffeine surface-displayed on the SLN facilitates receptor-mediated or receptor-adjacent interactions at the striatal tissue interface, augmenting particle retention and curcumin deposition in this region selectively relative to other brain areas.

A secondary observation merits brief notation: a rebound increase in curcumin concentrations was observed between the 30-minute and 60-minute time points across all three groups and all three matrices (plasma, striatum, and frontal cortex). This pattern is consistent with a secondary biodistribution route, most plausibly absorption of intranasally administered material via the gastrointestinal tract following inadvertent swallowing.

What is claimed is:

1. A composition for delivering an active pharmaceutical ingredient (API) and/or active nutraceutical ingredient (ANI) to a basal ganglia of a subject in need thereof comprising:
a plurality of nanoparticles surface decorated with caffeine, wherein said nanoparticles comprising: at least one API or ANI, at least one emulsifier, at least one emulsifier stabilizer, at least one water miscible solvent, and at least one solid lipid matrix core;
and
a pharmaceutically or nutraceutically acceptable carrier.

2. The composition of claim 1, wherein the emulsifier is PEG-40 hydrogenated castor oil (Cremophor RH40), polyoxyl 15 hydroxystearate, poloxamer 407 (Pluronic F127), lecithin (soy or sunflower, phosphatidylcholine content≥90%) and/or PEG-35 castor oil (Cremophor EL).

3. The composition of claim 1, wherein the emulsion stabilizer is polysorbate 20, sorbitan monooleate, polysorbate 80 (Tween 80), polysorbate 60 (Tween 60), sorbitan monostearate (Span 60), and/or sorbitan monolaurate (Span 20).

4. The composition of claim 1, wherein the water miscible solvent is propane-1,2-diol, polyethylene glycol 400 (PEG 400) and/or glycerol (glycerin), and/or 1,3-butanediol.

5. The composition of claim 1, wherein the solid lipid matrix core is cocoa butter, cetyl palmitate, glyceryl behenate (Compritol 888 ATO), glyceryl palmitostearate (Precirol ATO 5) and/or beeswax.

6. The composition of claim 1, wherein the nanoparticle is made of PEG-40 hydrogenated castor oil, polysorbate 20, sorbitan monooleate, polyoxyl 15 hydroxystearate, propane-1,2-diol and/or cocoa butter.

7. The composition of claim 6, wherein the amount of PEG-40 hydrogenated castor oil is between 10-30% w/w, the amount of polysorbate 20 is between 10-30% w/w, the amount of sorbitan monooleate is between 10-30% w/w, the amount of polyoxyl 15 hydroxystearate is between 2-15% w/w, the amount of propane-1,2-diol is between 10-35% w/w, and the amount of cocoa butter is between 8-20% w/w.

8. The composition of claim 6, wherein the amount of PEG-40 hydrogenated castor oil is between 12-25% w/w, the amount of polysorbate 20 is between 12-25% w/w, the amount of sorbitan monooleate is between 12-25% w/w, the amount of polyoxyl 15 hydroxystearate is between 2-10% w/w, the amount of propane-1,2-diol is between 15-28% w/w, and the amount of cocoa butter is between 10-20% w/w.

9. The composition of claim 8, wherein the amount of PEG-40 hydrogenated castor oil is between 15-20% w/w, the amount of polysorbate 20 is between 15-20% % w/w, the amount of sorbitan monooleate is between 15-20% w/w, the amount of polyoxyl 15 hydroxystearate is between 4-10% w/w, the amount of propane-1,2-diol is between 20-28% w/w, and the amount of cocoa butter is between 12-18% w/w.

10. The composition of claim 9, wherein the amount of PEG-40 hydrogenated castor oil about 18% w/w, the amount of polysorbate 20 is about 18% w/w, the amount of sorbitan monooleate is about 18% w/w, the amount of polyoxyl 15 hydroxystearate is about 6% w/w, the amount of propane-1,2-diol is about 22% w/w, and the amount of cocoa butter is about 14% w/w.

11. The composition of claim 1, wherein the API or the ANI is a lipophilic anti-inflammatory or antioxidant compound having a calculated Log P greater than 1.5 and a molecular weight of less than 900 Da.

12. The composition of claim 1, wherein the API or the ANI is curcumin, coenzyme Q10 (ubiquinone), resveratrol, baicalein, alpha-tocopherol, astaxanthin, quercetin, honokiol, pterostilbene, fisetin, tanshinone IIA, carnosic acid, luteolin, ursolic acid, nobiletin, thymoquinone, apigenin, piperine, withaferin A, andrographolide, and any combination thereof.

13. The composition of claim 1, wherein the API or the ANI is coenzyme Q10, resveratrol, baicalein, alpha-Tocopherol and/or astaxanthin.

14. The composition of claim 1, wherein the API or the ANI is curcumin.

15. The composition of claim 14, wherein the amount of the curcumin in the nanoparticle is between 1-8% w/w.

16. The composition of claim 15, wherein the amount of the curcumin in the nanoparticle is about 4% w/w.

17. A method of delivering API and/or ANI to the basal ganglia of a subject in need thereof comprising the step of intranasally administering the composition of claim 1 to the subject in need thereof.

18. The method of claim 17, wherein the API or ANI is curcumin.

19. A method of manufacturing the nanoparticles in the composition of claim 1 comprising:
mixing PEG-40 hydrogenated castor oil, polysorbate 20, sorbitan monooleate, polyoxyl 15 hydroxystearate, propane-1,2-diol, cocoa butter and the API and/or the ANI to form a plurality of nanoparticles comprising the API and/or the ANI; and
dispersing the plurality of nanoparticles in aqueous caffein dispersion.

20. A method of treating Parkinson's Disease comprising the step of intranasally administering the composition of claim 1 to a subject in need.
